(19) United States
(12) Patent Application Publication
(10) Pub. No.: US 2023/0108495 A1
(43) Pub. Date: Apr. 6, 2023

(54) RISK-STRATIFICATION OF MENINGIOMA PATIENTS

(71) Applicant: The Regents of the University of California, Oakland, CA (US)

(72) Inventors: David R. Raleigh, San Francisco, CA (US); William Chen, San Francisco, CA (US)

(21) Appl. No.: 17/906,299

(22) PCT Filed: Mar. 18, 2021
(86) PCT No.: PCT/US2021/070288

§ 371 Date: Sep. 14, 2022

Related U.S. Application Data
(60) Provisional application No. 62/991,486, filed on Mar. 18, 2020.

(51) Int. Cl. C12Q 1/6886; G01N 33/574

(52) U.S. Cl. CPC C12Q 1/6886; G01N 33/57492; C12Q 2600/158; C12Q 2600/112; C12Q 1/6837

(57) ABSTRACT

Provided herein is a panel of genes for predicting recurrence of meningioma and methods of using the expression pattern of the gene panel to provide a risk score for identifying patients for radiotherapy treatment.

### CROSS-REFERENCE TO RELATED APPLICATIONS

This application claims priority benefit of U.S. Provisional Application No. 62,991,486, filed Mar. 18, 2020, which is incorporated by reference in its entirety for all purposes.

### BACKGROUND OF THE INVENTION

Meningiomas constitute 38% of all primary intracranial tumors diagnosed in the United States, and are the most common tumor of the central nervous system¹. Many meningiomas are slow growing and can be cured with resection and/or radiotherapy; however, a significant subset have high World Health Organization (WHO) histopathologic grade, including atypical meningiomas (WHO grade II, 10-20%) and anaplastic meningiomas (WHO grade III, 3-5%), and are prone to local recurrence despite optimal local control¹. Moreover, there are subsets of patients with WHO grade I meningiomas who develop paradoxical recurrences that could not be predicted from histopathologic or clinical features²⁻⁵. Although many pathological, clinical, imaging and genomic prognostic factors have been investigated for meningioma⁶⁻¹¹, there are currently no standard or clinically tractable molecular criteria to identify meningiomas at risk for recurrence after resection. In parallel, the efficacy of adjuvant radiotherapy for meningioma is the topic of multiple ongoing prospective trials¹²⁻¹⁵, all of which stratify or randomize patients irrespective of molecular features that might help to identify patients in particular need of adjuvant treatment, or who could be spared from the added toxicity of ionizing radiation.

Recent efforts to characterize the genetic, transcriptional and epigenetic landscape of meningioma have been substantial. These efforts have identified certain mutually exclusive subgroups of meningiomas harboring recurrent mutations in TRAF7, KLF4, AKT1, and SMO, which almost exclusively occur in clinically indolent tumors¹⁶⁻¹⁹. Nevertheless, the majority of meningiomas, including nearly all WHO grade II and III meningiomas, do not appear to harbor recurrent genomic events beyond loss of chromosome 22 or inactivating mutations in the tumor suppressor NF2, with infrequent exceptions^{20,21}. Although high grade meningiomas are also characterized by widespread chromosomal instability with dramatic copy number variations (CNVs), the clinical and gene expression significance of most CNVs in high grade meningioma are poorly understood^{22,23}. Most recently, DNA methylation-based classification of meningiomas has emerged as a robust prognostic assay, albeit clinically-challenging test to implement in most centers^{7,16,23}. Indeed, DNA methylation-based classification of meningiomas appears to perform as well or slightly better than the WHO grading scheme for progression free survival^{16,21}, and equivalent to WHO grade for disease-specific survival. Whole genome transcriptomic profiling has also identified gene expression based subgroups of meningiomas that appear to stratify according to location and clinical outcomes^{10,23}, but like DNA methylation-based profiling, whole genome transcriptomic profiling of tumors remains challenging to implement clinically due to the financial, logistic and quality assurance burden of these approaches^{24,25}. It has also been shown that high meningioma cell proliferation in resection specimens identifies tumors at risk for adverse clinical outcomes^{3,26-28}, and that activation of the FOXM1 target genes drives meningioma cell proliferation across molecular subgroups and WHO grades²³.

There is an urgent unmet need for a clinically practical prognostic biomarker that could be used to distinguish high-risk meningioma patients who may benefit from adjuvant radiotherapy, and conversely, to spare low-risk meningioma patients from the potential toxicities of adjuvant treatment. Similar challenges in other cancer types have been met with the development of targeted gene expression based biomarkers that are now in widespread clinical use, particularly in breast cancer, where a 21-gene expression assay has been shown to be predictive of the need for adjuvant chemotherapy in a large randomized trial²⁹, and in prostate cancer, where similar gene expression assays are available to help risk-stratify patients and determine suitability for active surveillance^{30,31}.

### BRIEF SUMMARY

This section provides a summary of certain aspects of the disclosure. The invention is not limited to embodiments summarized in this section.

In one aspect, provided herein is a panel of biomarkers that provide a prognostic gene expression-based signature that allows the determination of a risk score for meningioma recurrence and methods of using the panel to assign a risk score for meningioma recurrence. Thus, in one aspect, provided herein is s method of evaluating the likelihood of recurrence of meningioma in a patient, the method comprising: detecting the levels of expression of each member of a panel of 36 genes, or a panel that comprises a subset of at least six genes of the 36-gene panel, in a sample from the patient that comprises meningioma tumor cells, wherein the 36 genes are: SFRP, NRAS, NQO1, COL1A1, CDC25C, MYBL2, CDC2, FOXM1, BIRC5, TOP2A, L1CAM, MMP9, SPP1, CXCL8, PIM1, PLAUR, IGF2, FLT1, KDR, AREG, NF2, FGR, CCND3, NDRG2, ERCC4, CCND2, BMI1, REL, MPL, BMP4, CYR61, CTGF, GAS1, IFNGR1, TMEM30B, and PGR; determining a normalized value for the level of expression of each member of the panel and assigning an expression score to each normalized value; summing the expression score for each gene to assign a risk score, wherein a high risk score in the top third tertile compared to a reference scale indicates that the patient has a high risk of local recurrence. In some embodiments, the subset comprises at least two genes from each of the following subgroups: Group 1, SFRP4, NRAS, NQO1, COL1A1, CDC25C, MYBL2, CDC2/CDK1, FOXM1, BIRC5, and TOP2A; Group 2, L1CAM, MMP9, SPP1, CXCL8, PIM1, PLAUR, and IGF2; and Group 3, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR. In some embodiments, the subset comprise a least three genes from each subgroup. In some embodiments, the subset comprise a least four genes from each subgroup. In some embodiments, the subset comprises at least one gene that is localized to chromosome arm 1p, at least one gene that is localized to chromosome arm 1q, at least one gene that is localized to chromosome arm 6q, at least one gene that is localized to chromosome arm 17q, and at least one gene that is localized to chromosome arm 20q. In other embodiments, the subset further comprises at least one gene that is localized to chromosome arm 3p, at least one gene that is localized to chromosome arm 7q, at least one gene that is localized to chromosome arm 11q, at least one gene that is localized to chromosome arm 14q, and at least one gene that is localized to chromosome arm 22q. In some embodiments, expression is detected by determining levels of RNA transcripts encoded by the genes, e.g., by performing an amplification assay, a hybridization assay, a sequencing assay or an array-based hybridization assay. In other embodiments, expression is detected by determining levels of proteins encoded by the genes, e.g., by performing an immunoassay. In some embodiments the reference scale is a plurality of risk scores derived from a population of reference patients that have meningioma. In some embodiments, the method further comprises recommending radiotherapy treatment to the patient when the patient has a high risk score. In some embodiments, the sample from the patient is a tumor tissue sample or a tumor cell sample.

In a further aspect, provided herein is a microarray comprising probes for detecting expression of a gene panel for predicting survival, wherein the gene panel is made up of the genes SFRP4, NRAS, NQO1, COL1A1, CDC25C, MYBL2, CDC2/CDK1, FOXM1, BIRC5, and TOP2A; Group 2, L1CAM MMP9, SPP1, CXCL8, PIM1, PLAUR, and IGF2; Group 3, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR, or a subset of at least 6 genes of this gene panel; and optionally contains probes for detecting expression of one or more reference genes, wherein the microarray contains probes for detecting no more than 200 genes, or no more than 100 genes. In some embodiments, the subset comprise at least two genes from each of the following subgroups: Group 1, SFRP4, NRAS, NQO1, COL1A1, CDC25C, MYBL2, CDC2/CDK1, FOXM1, BIRC5, and TOP2A; Group 2, L1CAM MMP9, SPP1, CXCL8, PIM1, PLAUR, and IGF2; Group 3, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR. In some embodiments, the subset comprises at least three genes from each of the subgroups. In some embodiments, the subset comprises at least four genes from each of the subgroups. In some embodiments, the subset comprises at least one gene that is localized to chromosome arm 1p, at least one gene that is localized to chromosome arm 1q, at least one gene that is localized to chromosome arm 6q, at least one gene that is localized to chromosome arm 17q, and at least one gene that is localized to chromosome arm 20q. In further embodiments, the subset further comprises at least one gene that is localized to chromosome arm 3p, at least one gene that is localized to chromosome arm 7q, at least one gene that is localized to chromosome arm 11q, at least one gene that is localized to chromosome arm 14q, and at least one gene that is localized to chromosome arm 22q.

In a further aspect, provided herein is a kit comprising primers and/or probes for detecting expression of a gene panel for predicting survival, wherein the gene panel consists of the gene SFRP, NRAS, NQO1, COL1A1, CDC25C, MYBL2, CDC2, FOXM1, BIRC5, TOP2A, L1CAM, MMP9, SPP1, CXCL8, PIM1, PLAUR, IGF2, FLT1, KDR, AREG, NF2, FGR, CCND3, NDRG2, ERCC4, CCND2, BMI1, REL, MPL, BMP4, CYR61, CTGF, GAS1, IFNGR1, TMEM30B, and PGR, or a subset of at least 6 genes of this gene panel, and optionally contains primers and/or probes for detecting expression of one or more reference genes. In some embodiments, the subset comprise at least two genes from each of the following subgroups: Group 1, SFRP4, NRAS, NQO1, COL1A1, CDC25C, MYBL2, CDC2/CDK1, FOXM1, BIRC5, and TOP2A; Group 2, L1CAM MMP9, SPP1, CXCL8, PIM1, PLAUR, and IGF2; Group 3, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR. In some embodiments, the subset comprises at least three genes from each of the subgroups. In some embodiments, the subset comprises at least four genes from each of the subgroups. In additional embodiment, the subset comprises at least one gene that is localized to chromosome arm 1p, at least one gene that is localized to chromosome arm 1q, at least one gene that is localized to chromosome arm 6q, at least one gene that is localized to chromosome arm 17q, and at least one gene that is localized to chromosome arm 20q. In some embodiments, the subset further comprises at least one gene that is localized to chromosome arm 3p, at least one gene that is localized to chromosome arm 7q, at least one gene that is localized to chromosome arm 11q, at least one gene that is localized to chromosome arm 14q, and at least one gene that is localized to chromosome arm 22q.

In another aspect, provided herein is a panel of biomarkers that provide a prognostic gene expression-based signature that allows the determination of a risk score for meningioma recurrence and methods of using the panel to assign a risk score for meningioma recurrence. Thus, in one aspect, provided herein is s method of evaluating the likelihood of recurrence of meningioma in a patient, the method comprising: detecting the levels of expression of each member of a panel of 34 genes or a panel that comprises a subset of at least eight genes of the 34-gene panel, in a sample from the patient that comprises meningioma tumor cells, wherein the 34 genes are: ARID1B, CCL21, CCN1, CCND2, CD3E, CDC20, CDK6, CDKN2A, CDKN2C, CHEK1, CKS2, COL1A1, ESR1, EZH2, FBLIM1, FGFR4, GAS1, IFNGR1, IGF2, KDR, KIF20A, KRT14, LINC02593, MDM4, MMP9. MUTYH, MYBL1, PGK1, PGR, PIM1, SPOP, TAGLN, TMEM30B, and USF1; determining a normalized value for the level of expression of each member of the panel and assigning an expression score to each normalized value; summing the expression score for each gene to assign a risk score, wherein a high risk score in the top third tertile compared to a reference scale indicates that the patient has a high risk of local recurrence. In some embodiments, a subset comprises at least one gene from each of the following Groups 1-7; or at least two genes from each of Groups 1-3 and optionally, at least two genes selected from the genes listed in Groups 4-7 (CHEK1, MUTYH; PGR, ESR; LINC02593, FBLIM1; CCL21 and CD3E): Group 1, CDC20, CDK6, CCND2, CKS2, MYBL1, USF1, KIF20A, MDM4, and PIM1; Group 2, CDKN2A, CDKN2C, ARID1B, GAS1, and SPOP; and Group 3, CCN1, COL1A1, FGFR4, IFNGR1, IGF2, KDR, KRT14, MMP9, TAGLN, TMEM30B, and PGK1; Group 4, CHEK1 and MUTYH; Group 5, PGR and ESR; Group 6, LINC02593 and FBLIM1; and Group 7, CCL21 and CD3E. In some embodiments, expression is detected by determining levels of RNA transcripts encoded by the genes, e.g., by performing an amplification assay, a hybridization assay, a sequencing assay or an array-based hybridization assay. In other embodiments, expression is detected by determining levels of proteins encoded by the genes, e.g., by performing an immunoassay. In some embodiments the reference scale is a plurality of risk scores derived from a population of reference patients that have meningioma. In some embodiments, the method further comprises recommending radiotherapy treatment to the patient when the patient has a high risk score. In some embodiments, the sample from the patient is a tumor tissue sample or a tumor cell sample.

In a further aspect, provided herein is a microarray comprising probes for detecting expression of a gene panel for predicting survival, wherein the gene panel consists of the gene ARID1B, CCL21, CCN1, CCND2, CD3E, CDC20, CDK6, CDKN2A, CDKN2C, CHEK1, CKS2, COL1A1, ESR1, EZH2, FBLIM1, FGFR4, GAS1, IFNGR1, IGF2, KDR, KIF20A, KRT14, LINC02593, MDM4, MMP9. MUTYH, MYBL1, PGK1. PGR, PIM1, SPOP. TAGLN, TMEM30B, and USF1, or a subset of at least eight genes of the gene panel; and optionally contains probes for detecting expression of one or more reference genes, wherein the microarray contains probes for detecting no more than 1,000 genes, no more than 500 genes, no more than 200 genes, or no more than 100 genes. In some embodiments, a subset comprises at least one gene from each of the following Groups 1-7; or at least two genes from each of Groups 1-3 and optionally, at least two genes selected from the genes listed in Groups 4-7 (CHEK1, MUTYH; PGR, ESR; LINC02593, FBLIM1; CCL21 and CD3E): Group 1, CDC20, CDK6, CCND2, CKS2, MYBL1, USF1, KIF20A, MDM4, and PIM1; Group 2, CDKN2A, CDKN2C, ARID1B, GAS1, and SPOP; and Group 3, CCN1, COL1A1, FGFR4, IFNGR1, IGF2, KDR, KRT14, MMP9, TAGLN, TMEM30B, and PGK1; Group 4, CHEK1 and MUTYH; Group 5, PGR and ESR; Group 6, LINC02593 and FBLIM1; and Group 7, CCL21 and CD3E.

In a further aspect, provided herein is a kit comprising primers and/or probes for detecting expression of a gene panel for predicting survival, wherein the gene panel consists of the gene ARID1B, CCL21, CCN1, CCND2, CD3E, CDC20, CDK6, CDKN2A, CDKN2C, CHEK1, CKS2, COL1A1, ESR1, EZH2, FBLIM1, FGFR4, GAS1, IFNGR1, IGF2, KDR, KIF20A, KRT14, LINC02593, MDM4, MMP9. MUTYH, MYBL1, PGK1. PGR, PIM1, SPOP. TAGLN, TMEM30B, and USF1, or a subset of at least eight genes of the gene panel, and optionally contains primers and/or probes for detecting expression of one or more reference genes. In some embodiments, a subset comprises at least one gene from each of the following Groups 1-7; or at least two genes from each of Groups 1-3 and optionally, at least two genes selected from the genes listed in Groups 4-7 (CHEK1, MUTYH; PGR, ESR; LINC02593, FBLIM1; CCL21 and CD3E):

Group 1, CDC20, CDK6, CCND2, CKS2, MYBL1, USF1, KIF20A, MDM4, and PIM1; Group 2, CDKN2A, CDKN2C, ARID1B, GAS1, and SPOP; and Group 3, CCN1, COL1A1, FGFR4, IFNGR1, IGF2, KDR, KRT14, MMP9, TAGLN, TMEM30B, and PGK1; Group 4, CHEK1 and MUTYH; Group 5, PGR and ESR; Group 6, LINC02593 and FBLIM1; and Group 7, CCL21 and CD3E.

Other objects, features, and advantages of the present invention will be apparent to one of skill in the art from the following detailed description and figures.

### BRIEF DESCRIPTION OF THE DRAWINGS

FIG. 1A-F. Targeted gene expression analysis of clinically aggressive meningiomas identifies a prognostic gene signature that outperforms WHO grade. A) Unsupervised hierarchical clustering of prognostic genes identified using prediction analysis for microarrays (PAM) confirms the ability of the gene set to stratify meningioma patients into high risk (red cluster) and lower risk categories (blue cluster, Log-rank test, p<0.0001). Gene expression is normalized by row. B) Gene enrichment analysis of prognostic gene clusters from A) identifies a tightly correlated set of genes involved in cell-cycle processes (orange cluster), and clusters of genes involved in cellular signaling and extracellular matrix interactions (light blue and grey clusters). C) Representative IHC images demonstrating high TMEM30B staining on the top right (20× magnification) and low/absent TMEM3B staining on the top left. Similarly, representative IHC images demonstrating low SFRP4 staining (20× magnification) on the bottom left and high SFRP4 staining on the bottom right are shown. Low TMEM30B staining (15 of 96 meningiomas, 16%) is associated with a trend towards worse LFFR, and high SFRP4 staining (46 of 94 meningiomas, 49%) is significantly associated with worse LFFR. D) Elastic net regression was used to generate a gene signature risk score between 0 and 1 per tumor sample (Accuracy 0.80, AUC 0.86). Gene risk score correlates with tumor grade and is correlated with a faster time to failure (Time to failure vs log(gene risk), p<0.0001, F-test). Meningiomas with a gene risk score of greater than 0.5 uniformly recur within 2 years of resection. E) The gene signature risk score outperforms WHO grade in stratifying LFFR (P<0.001 vs P=0.09, Log-rank test) and OS (P<0.0001 vs P=0.07, Log-rank test). F) After adjusting for age, sex, extent of resection, and grade using multivariate Cox regression, the gene signature risk score is independently associated with recurrence (RR 1.56 per 0.1 risk score increase, 95% CI 1.30-1.90) and mortality (RR 1.32 per 0.1 increase, 95% CI 1.07-1.64). After stratifying patients by grade, the gene signature risk score remains significantly prognostic for meningioma recurrence and mortality on univariate Cox regression. Further, among gross totally resected grade 2 tumors (Grade 2+GTR), the gene risk score is significantly prognostic of recurrence.

FIG. 2A-C. Prognostic gene signature risk score validation in an independent dataset outperforms WHO grade in prognosticating meningioma patient survival. A) Meningioma gene signature risk scores were calculated on an independent validation dataset from an outside institution. The gene signature risk score remains correlated with WHO grade and with faster time to failure (TTF) among patients who recurred (TTF vs log(GS risk), p=0.002, F-test). B) The gene risk score remains significantly associated with worse LFFR (P=0.0004, Log-rank test) and outperforms WHO grade in stratifying patients by overall survival (P=0.003 vs P=0.10, Log-rank test). C) The gene signature risk score remains significantly prognostic for mortality (RR 1.86 per 0.1 increase, 95% CI 1.19-2.88) after adjusting for WHO grade on Cox regression.

FIG. 3A-C. Analysis of chromosome locations of prognostic genes identifies areas of frequent amplification or deletion associated with aggressive meningioma, and identifies a core set of signature genes highly correlated with copy number variations.

A) All 266 genes from the nanostring discovery dataset are displayed by chromosome location. A moving average of neighboring gene-gene correlation (ρ, window size 4 genes) identified chromosome regions with highly co-expressed genes corresponding to areas of known frequent CNVs in meningioma, including 1p, 1q, 3p, 6q, 7q, 11q, 14q, 17q, 20q, and 22q. Coefficients of univariate Cox regression between gene expression and local recurrence are displayed (β, color-scale −3 to 3), as well as p-values (color-scale 0.05 to 0). Areas of negative β, shown in blue, correspond to areas where presumed CNV deletions are associated with worse outcome, and areas of positive β, shown in red, correspond to areas where presumed CNV amplifications are associated with worse outcome. Multiple genes from the prognostic gene signature appear to cluster in the 1p, 1q, 6q, 17q, and 20q regions, although most prognostic genes exist in areas of low neighboring-gene correlation, which may represent conserved areas infrequently affected by CNV. B) Analysis of the total number of CNVs and gene expression in the validation microarray cohort identified 397 genes significantly correlated with CNV number (FDR q-value<0.05). Four gene signature genes were among these: FOXM1, CDC25C, TOP2A, and BIRC5, which form a tightly co-expressed gene network highly correlated with CNV number (p<0.0001, F-test). C) STRING protein-protein interaction analysis and clustering of prognostic genes (confidence level threshold 0.7, MCF clustering, inflation parameter=3) yielded a cluster of proliferative genes (red) containing these CNV-correlated genes: FOXM1, CDC25C, TOP2A, and CDC25C, and a cluster of mesenchymal genes involved in osteoblast differentiation and collagen development (yellow).

FIG. 4A-D. Meningioma gene expression is prognostic for meningioma outcomes. A) Distribution of the targeted gene expression risk score in the discovery cohort is shown. The risk score varies between 0 and 1, with higher risk score correlating with faster time to recurrence. B) Kaplan-Meier curves for local freedom from recurrence (left) and overall survival (right) stratified by gene expression risk score showed strong prognostic discrimination in the discovery cohort (top, middle, and lower curves correspond to low, intermediate, and high risks). C) When the locked model and thresholds were applied to the validation cohort (n=331), the risk score remained well distributed between 0 and 1, and retained its correlation with faster time to recurrence. D) Kaplan-Meier curves demonstrate strong prognostic discrimination between risk groups based upon the gene expression risk score in the validation cohort.

FIG. 5. Meningioma gene expression is independently prognostic for local control and survival. Forest plots for hazard ratios and 95% confidence intervals are shown for univariate or multivariate Cox regression for the targeted gene expression risk score across clinical contexts (top: clinical contexts, middle: common copy number variant subgroups, bottom, methylation groups and multivariate regression) for both endpoints of LFFR and OS, and for both the discovery and validation cohorts, demonstrating its independent prognostic value. The grade adjusted hazard ratios represent a Cox model adjusting for WHO grade, and the multivariate hazard ratios in the last row represents a Cox model adjusting for all the variables above, including WHO grade, extent of resection, copy number variation status (Ch1p and Ch22q), methylation group, and, for the OS model, age.

FIG. 6A-C. Targeted meningioma gene expression profiling predicts radiotherapy responses. A) Kaplan-Meier curves for LFFR are shown for the combined cohorts of WHO grade 2 meningiomas, as stratified by receipt of adjuvant radiotherapy and gene expression risk strata: low vs intermediate/high risk; intermediate/high risk patients experienced improved LFFR with receipt of radiotherapy (HR 0.54, 95% confidence interval 0.3-1.0, p=0.0495), while low risk patients did not (HR 1.0, 95% confidence interval 0.2-7.2, p=0.9690). B) Similar predictive value was observed for the gene expression risk score for a propensity matched cohort of patients of all WHO grades who did or did not receive adjuvant radiotherapy, matched on a comprehensive set of covariates including WHO grade, extent of resection, setting, methylation group, copy number alteration status, and MIB labeling index. C) Stratification of the validation cohort by RTOG 0539 criteria for risk stratification and receipt of radiotherapy suggested that application of the targeted gene expression risk score would result in change in management of 32% of RTOG 0539 low risk, 35% of RTOG 0539 intermediate risk, and 9% of RTOG 0539 high risk meninigiomas (30.2% of cases total)

FIG. 7A-E. Targeted meningioma gene expression profiling provides improved outcomes discrimination. A) AUC for LFFR at 5 years in the validation cohort is shown here for WHO grade, DNA methylation group, and the gene expression risk score (both continuous and divided by low, intermediate, and high risk), with the gene expression risk score achieving significantly higher AUC (0.81) compared to WHO grade (0.67). B) Brier error scores are shown for the same groups, demonstrating that the gene expression risk score achieves the lowest model error across all time points (integrated Brier error 0.14). C) Comprehensive models incorporating clinical covariates (WHO grade, setting, extent of resection, adjuvant radiation) with or without the addition of the targeted gene expression risk score or methylation group are shown, demonstrating the additive benefit of the targeted gene expression risk score. D) Brier error scores are shown for the above, again showing that the combined model with gene expression risk score achieves lower error across all time points compared to WHO grade. E) A nomogram based upon Cox regression with the covariates shown is displayed for estimation of 5-year LFFR, and demonstrating the dominant role of the gene expression risk score in determining this risk.

FIG. 8. Model and gene selection for meningioma freedom from local progression. Concordance index is plotted against the log of the lambda parameter with performance and error estimated by 10-fold cross validation, resulting in an optimal model chosen with a model of minimal size but still within 1 standard error of the model achieving maximal c-index (bordered by dotted lines). This model contained the 34 genes used in the subsequent analyses.

FIG. 9A-D. Discovery cohort characteristics. Characteristics and representative Kaplan-Meier curves are shown for the discovery cohort.

FIG. 10A-D. Validation cohort characteristics. Characteristics and representative Kaplan-Meier curves are shown for the validation cohort.

FIG. 11. Targeted meningioma gene expression profiling is prognostic across WHO grades. Characteristics Kaplan Meier curves are shown for the validation cohort in selected clinically relevant contexts. In particular, the gene expression risk score remains prognostic in WHO grade 1 tumors, WHO grade 1 tumors after gross total resection, as well as in higher grade tumor subgroups.

FIG. 12 Targeted meningioma gene expression profiling is prognostic across DNA methylation groups. The gene expression risk score remains prognostic within the immune-enriched and hypermitotic methylation groups, within the validation cohort.

FIG. 13A-B Targeted meningioma gene expression profiling is prognostic for disease-specific survival. The gene expression risk score was prognostic for disease specific survival in the A) discovery and B) validation cohorts.

FIG. 14A-D Meningioma WHO grade or DNA methylation group does not predict radiotherapy responses. Neither A-B) WHO grade, methylation group C), or the combination D), were predictive for radiotherapy response in the combined cohort.

FIG. 15. Multivariable Cox regression outputs in the validation dataset. Hazard ratios and 95% confidence intervals are shown for Cox multivariable regression within the validation cohort.

FIG. 16. Calibration curve for clinical nomogram model in the validation dataset. A calibration curve is shown for 5-year LFFR with the final clinical+gene expression risk score model, as estimated by bootstrap resampling with B=1000 iterations.

### DETAILED DESCRIPTION

Described herein are methods and compositions for predicting the risk of meningioma recurrence following resection. The method includes determining the expression level, such as the RNA expression level or the protein expression level of a panel of 36 genes, i.e., SFRP4, NRAS, NQO1, COL1A1, CDC25C, MYBL2, CDC2/CDK1, FOXM1, BIRC5, TOP2A, L1CAM, MMP9, SPP1, CXCL8, PIM1, PLAUR, IGF2, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR, or a subset thereof that includes at least six genes, as described herein, transforming the levels into a risk score, and determining that the subject has a likelihood of recurrence if the risk score is high. In some instances, a high risk represents any value in the top tertile of a reference range of values. In other instances, a high risk may represent values above a threshold calibrated to the top tertile of risk of recurrence.

In some embodiments, the disclosure provides method and compositions for predicting risk of meningioma using a method comprising determining the expression level, such as the RNA expression level or the protein expression level of a panel of 34 genes, i.e., ARID1B, CCL21, CCN1, CCND2, CD3E, CDC20, CDK6, CDKN2A, CDKN2C, CHEK1, CKS2, COL1A1, ESR1, EZH2, FBLIM1, FGFR4, GAS1, IFNGR1, IGF2, KDR, KIF20A, KRT14, LINC02593, MDM4, MMP9. MUTYH, MYBL1, PGK1. PGR, PIM1, SPOP. TAGLN, TMEM30B, and USF1, or a subset thereof that includes at least eight genes, transforming the levels into a risk score, and determining that the subject has a likelihood of recurrence if the risk score is high. In some instances, a high risk represents any value in the top tertile of a reference range of values. In other instances, a high risk may represent values above a threshold calibrated to the top tertile of risk of recurrence

### Terminology

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “meningioma sample” includes any biological sample that contains meningioma tumor cells. Biological samples include samples obtained from body fluids, e.g., blood, plasma, serum, or urine; or samples derived, e.g., by biopsy, from cells, tissues or organs, preferably tumor tissue comprising meningioma tumor cells.

The terms “determining,” “assessing,” “assaying,” “measuring” and “detecting” can be used interchangeably and refer to quantitative determinations.

The term “amount” or “level” refers to the quantity of a polynucleotide of interest or a polypeptide of interest present in a sample. Such quantity may be expressed as the total quantity of the polynucleotide or polypeptide in the sample, in relative terms, as a concentration of the polynucleotide or polypeptide in the sample, or as a relative quantity compared to a reference value.

As used herein, the term “expression level” of a gene as described herein refers to the level of expression of an RNA transcript of the gene or the level of polypeptide translation product. The term “normalized level” or “normalized expression level” of a gene refers to the level of expression of the RNA transcript or polypeptide translation product after normalization based on the expression levels of one or more reference genes, e.g., a constitutively expressed gene.

As used herein “an RNA” measured in accordance with the invention refers to any RNA encoded by the gene, including, for example, mRNA, splice variants, unspliced RNA, fragments, or microRNA.

Genes are referred to herein using the official symbol and official nomenclature for the human gene as assigned by the HUGO Gene Nomenclature Committee (HGNC). In the present disclosure, an individual gene as designated herein may also have alternative designations, e.g., as indicated in the HGNC database as of the filing date of the present application. For example, CDK1 is also known as CDC2, CDC28A, or P34CDC2; CCN1 is also known as CYR61 or IGFBP10; and CCN2 is also known as CTGF or IGFBP8. As used herein, the term “signature gene” refers to a gene whose expression is correlated, either positively or negatively, with meningioma recurrence. A “signature gene panel” is a collection of such signature genes for which the gene expression scores are generated and used together to provide a risk score for meningioma recurrence. Thus, for example, a 36-gene signature panel of the panel, or a subset thereof as described herein, includes the following genes, the listing includes the human chromosomal localization in parenthesis following the gene designation as shown in the HGNC database as of the priority date of this application: SFRP4 (7p14.1), NRAS (1p13.2), NQO1 (16q22.1), COL1A1 (17q21.33), CDC25C (5q31.2), MYBL2 (20q13.12), CDC2/CDK1 (10q21.2), FOXM1 (12p13.33), BIRC5 (17q25.3), TOP2A (17q21.2), L1CAM (Xq28), MMP9 (20q13.12), SPP1 (4q22.1), CXCL8 (4q13.3), PIM1 (6p21.2), PLAUR (19q13), IGF2 (11p15.5), FLT1 (13q12.3), KDR (4q12), AREG (4q13.3), NF2 (22q12.2), FGR (1p35.3), CCND3 (6p21.1), NDRG2 (14q11.2), ERCC4 (16p13.12), CCND2 (12p13.32), BMI1 (10p12.2), REL (2p16.1), MPL (1q34.2), BMP4 (14q22.2), CYR61/CCN1 (1p22.3), CTGF/CCN2 (6q23.2), GAS1 (9q21.33), IFNGR1 (6q23.3), TMEM30B (14q23.1), and PGR (11q22.1). As a further example, a 34-gene signature panel includes the following genes: ARID1B (6q25.3), CCL21 9p13.3), CCN1 (1p22.3), CCND2 (12p13.32), CD3E (11q23.3), CDC20 (1p34.2), CDK6 (7q21.2), CDKN2A (9p21.3), CDKN2C (1p32.3), CHEK1 (11q24.2), CKS2 (9q22.2), COL1A1 (17q21.33), ESR1 (6q25.1), EZH2 (7q36.1), FBLIM1 (1p36.21), FGFR4 (5q35.2), GAS1 (9q21.33), IFNGR1 (6g23.3), IGF2 (11p15.5), KDR (4q12), KIF20A (5q31.2), KRT14 (17q21.2), LINC02593 (1p36.33), MDM4 (1q32.1), MMP9 (20q13.12). MUTYH (1p34.1), MYBL1 (8q13.1), PGK1 (Xq21.1). PGR (11q22.1), PIM1 (6p21.2), SPOP (17q21.33). TAGLN (11q23.3), TMEM30B (14q23.1), and USF1 (1q23.3). Reference to the gene by name includes any allelic variant or splice variants, that are encoded by the gene.

As used herein, “recurrence” refers to both local recurrence or recurrence at another site, e.g., at a metastatic site. “Recurrence” in this context, is an indicator of aggressiveness of the tumor.

The term “microarray” refers to an ordered arrangement of hybridizable array elements, e.g. oligonucleotide or polynucleotide probes, on a substrate.

The term “nucleic acid” or “polynucleotide” as used herein refers to a deoxyribonucleotide or ribonucleotide in either single- or double-stranded form. The term encompasses nucleic acids containing known analogues of natural nucleotides which have similar or improved binding properties, for the purposes desired, as the reference nucleic acid. The term also includes nucleic acids which are metabolized in a manner similar to naturally occurring nucleotides or at rates that are improved for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described in WO 97/03211; WO 96/39154; Mata (1997) Toxicol. Appl. Pharmacol. 144:189-197. Other synthetic backbones encompassed by the term include methyl-phosphonate linkages or alternating methylphosphonate and phosphodiester linkages (Strauss-Soukup (1997) Biochemistry 36: 8692-8698), and benzylphosphonate linkages (Samstag (1996) Antisense Nucleic Acid Drug Dev 6: 153-156).

The term “protein,” “peptide” or “polypeptide” are used interchangeably herein to refer to a polymer of amino acid residues. In the context of analysis of the levels of proteins encoded by signatures genes, the terms refer to naturally occurring amino acids linked by covalent peptide bonds. In a broader context, the terms can apply to amino acid polymers in which one or more amino acid residue is an artificial amino acid mimetic of a corresponding naturally occurring amino acid and/or the peptide chain comprises a non-naturally occurring bond to link the residues.

The term “gene product” or “gene expression product” refers to an RNA or protein encoded by the gene.

The term “hybridizing” refers to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences in a mixed population (e.g., RNA prepared from a tissue biopsy). “Stringency” of hybridization reactions is readily determinable by one of ordinary skill in the art, and generally is an empirical calculation dependent upon probe sequence, probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. Guidance for determining hybridization conditions for nucleic acids can be found in any number of well-known manuals, e.g., Current Protocols in Molecular Biology (K. Adelman, et al. eds., (John Wiley & Sons, 1987-through March 2020).

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

The term “treatment,” “treat,” or “treating” typically refers to a clinical intervention to ameliorate at least one symptom of a disease or otherwise slow disease progression. This includes preventing or slowing recurrence of the disease or metastasis of the disease, alleviation of symptoms, diminishment of any direct or indirect pathological consequences of the disease, amelioration or palliation of the disease state, and remission or improved prognosis. In some embodiments, the treatment may increase overall survival. In some instances, the treatment may increase overall survival (OS) (e.g., by about 5% or greater, about 10% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, or about 99% or greater). In some instances, the treatment may increase progression-free survival (PFS) (e.g., by about 5% or greater, about 10% or greater, about 20% or greater, about 25% or greater, about 30% or greater, about 35% or greater, about 40% or greater, about 45% or greater, about 50% or greater, about 55% or greater, about 60% or greater, about 65% or greater, about 70% or greater, about 75% or greater, about 80% or greater, about 85% or greater, about 90% or greater, about 95% or greater, about 96% or greater, about 97% or greater, about 98% or greater, or about 99% or greater). It is understood that treatment does not necessarily refer to a cure or complete ablation of the disease, condition, or symptoms of the disease or condition. In some embodiments, for example, for a patient that has a meningioma that has a low risk or recurrence, a “treatment” includes active surveillance to monitor the patients for recurrence of the tumor.

The term “recommending” or “suggesting,” in the context of a treatment of a disease, refers to making a suggestion or a recommendation for therapeutic intervention (e.g., radiotherapy, etc.) and/or disease management which are specifically applicable to the patient.

The term “subject” or “patient” is intended to include animals. Examples of subjects include mammals, e.g., humans, dogs, cows, horses, pigs, sheep, goats, cats, mice, rabbits, rats, and transgenic non-human animals. In preferred embodiments, the subject is a human that has meningioma.

The term “risk score” refers to a statistically derived value that can provide physicians and caregivers valuable diagnostic and prognostic insight. In some instances, the score provides a projected risk of recurrence. An individual's score can be compared to a reference score or a reference score scale to determine risk of disease recurrence/relapse or to assist in the selection of therapeutic intervention or disease management approaches.

The term “high risk score,” refers to an expression score generated from the normalized expression values of each member of the 36-gene panel described herein, or a subset of at least six genes in the panel, having a numerical value in the top percentile range, such as the top tertile (e.g., top 33%) of a range of risk scores for recurrence in meningioma patients. A “low risk score” refers to a value in the bottom percentile range, such as the lower tertile of the range. Similarly, “high risk score,” refers to an expression score generated from the normalized expression values of each member of the 34-gene panel described herein, or a subset of at least eight genes in the panel, having a numerical value in the top percentile range, such as the top tertile (e.g., top 33%), of a range of risk scores for recurrence in meningioma patients. A “low risk score” refers to a value in the bottom percentile range, such as the lower tertile of the range

### Gene Signature Panel

The methods described herein are based, in part, on the identification of a panel of 36 genes that collectively provide a risk score for meningioma recurrence in patients following resection based on normalized expression levels. The 36 genes are: SFRP4, NRAS, NQO1, COL1A1, CDC25C, MYBL2, CDC2/CDK1, FOXM1, BIRC5, TOP2A, L1CAM MMP9, SPP1, CXCL8, PIM1, PLAUR, IGF2, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR. Reference to “the 36-gene panel” in this disclosure refers to this panel of genes unless otherwise indicated. In other instances, a high risk may represent values above a threshold calibrated to the top tertile of risk of recurrence. In some embodiments, the expression levels, e.g., RNA expression levels of each of the 36 genes in the panel are evaluated in a sample from a meningioma and combined to generate a predictive score for recurrence. The meningioma sample may be obtained prior to, or during surgery. In some embodiments, the meningioma is a WHO grade I or WHO grade II meningioma, where the grade is determined based on the criteria of the most recent WHO guidelines for meningioma grading as of the filing date of this application.

In another aspect, the methods described herein are based, in part, on the identification of a panel of 34 genes, or a subset thereof, that collectively provide a risk score for meningioma recurrence in patients following resection based on normalized expression levels. The 34 genes are: ARID1B, CCL21, CCN1, CCND2, CD3E, CDC20, CDK6, CDKN2A, CDKN2C, CHEK1, CKS2, COL1A1, ESR1, EZH2, FBLIM1, FGFR4, GAS1, IFNGR1, IGF2, KDR, KIF20A, KRT14, LINC02593, MDM4, MMP9. MUTYH, MYBL1, PGK1. PGR, PIM1, SPOP. TAGLN, TMEM30B, and USF1. Reference to “the 34-gene panel” in this disclosure refers to this panel of genes unless otherwise indicated. In other instances, a high risk may represent values above a threshold calibrated to the top tertile of risk of recurrence. In some embodiments, the expression levels, e.g., RNA expression levels, of each of the 34 genes in the panel are evaluated in a sample from a meningioma and combined to generate a predictive score for recurrence. The meningioma sample may be obtained prior to, or during surgery. In some embodiments, the meningioma is a WHO grade I or WHO grade II meningioma, where the grade is determined based on the criteria of the most recent WHO guidelines for meningioma grading as of the filing date of this application.

In other embodiments, normalized expression levels, e.g., RNA expression, of a subset of 6 or more genes of the 36-gene panel are determined to generate a predictive score for recurrence, wherein the 6 or more genes comprise at least 2 genes from each of the following three subgroups of the 36 genes in the panel: Group 1, SFRP4, NRAS, NQO1, COL1A1, CDC25C, MYBL2, CDC2/CDK1, FOXM1, BIRC5, TOP2A; Group 2, L1CAM, MMP9, SPP1, CXCL8, PIM1, PLAUR, IGF2; Group 3, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR. In some embodiments, the panel comprises at least three genes from one of the subgroups and at least two or three genes from each of the other subgroups. In some embodiments, the gene panel comprises three genes from each of the subgroups. In some embodiments, the gene panel comprises at least four genes from one of the subgroups; and at least two or three genes from from each of the other subgroups. In some embodiments, the gene panel comprises four genes from each of the subgroups. In some embodiments, the gene panel comprises FOXM1, CDC25C, TOP2A, BIRC5, and at least two genes from the two other subgroups. In some embodiments, the gene panel comprises a subset of at least 18 genes or at least 24 genes from the 36-gene panel.

In other embodiments, normalized expression levels, e.g., RNA expression, of a subset of 10 or more genes of the 36-gene panel are determined to generate a predictive score for recurrence, wherein the 10 or more genes comprise at least 1 or 2 genes from each of the following subgroups of the 36 genes in the panel, wherein the subgroups are designated by the chromosomal arm: 1p (FGR, MPL, CYR61/CCN1, NRAS), 1q (MPL), 6q (CTGF/CCN2, IFNGR1), 14q (TMEM30B), 17q (TOP2A, COL1A1, BIRC5), and 20q (MYBL2). In some embodiments, the panel comprises at least three genes from one of the subgroups and at least two or three genes from each of the other subgroups. In some embodiments, the gene panel comprises three genes from each of the subgroups. In some embodiments, the gene panel comprises at least four genes from one of the subgroups; and at least two, three or four genes from from each of the other subgroups. In some embodiments, the gene panel comprises FOXM1, CDC25C, TOP2A, BIRC5, in addition to 1 or more genes from each of the subgroups designated by chromosomal arm.

In some embodiments, the gene panel comprises a subset of at least 11, 12, 13, 14, 15, 16, 17, or 18 genes of the 36-gene panel. In some embodiments, the panel comprises a subset of at least 19, 20, 21, 22, 23, or 24 genes of the 36-gene panel. In some embodiments, the gene panel comprises a subset of at least 25, 26, 27, 28, 29, or 30 genes of the 36-gene panel. In some embodiments, the gene panel comprises a subset of 31, 32, 33, 34, or 35 genes of the 36-gene panel. In typical embodiments, the gene panel comprises all of the genes of the 36-gene panel.

In other embodiments, normalized expression levels, e.g., RNA expression, of a subset of eight or more genes of the 34-gene panel are determined to generate a predictive score for recurrence, wherein the eight or more genes comprise at least 2 genes from each of the following Groups 1-3 of the 34 genes in the panel; at least two genes selected from the genes listed in Groups 4-7: Group 1, CDC20, CDK6, CCND2, CKS2, MYBL1, USF1, KIF20A, MDM4, and PIM1; Group 2, CDKN2A, CDKN2C, ARID1B, GAS1, and SPOP; and Group 3, CCN1, COL1A1, FGFR4, IFNGR1, IGF2, KDR, KRT14, MMP9, TAGLN, TMEM30B, and PGK1; Group 4, CHEK1 and MUTYH; Group 5, PGR and ESR; Group 6, LINC02593 and FBLIM1; and Group 7, CCL21 and CD3E. In some embodiments, normalized expression levels, e.g., RNA expression, is determined for a panel comprising a subset of 10 or more genes of the 34-gene panel. In some embodiments, the panel comprises a subset of 15 or more genes of the 34-gene panel; or a subset of 20 or more genes of the 34-gene panel; or a subset of 25 or more genes of the 34 gene-panel. In some embodiments, the method comprises determining normalized expression levels, e.g., RNA expression, for the genes in each of the subsets and to at least one gene listed in Table 5

In some embodiments, a gene panel evaluated to assess risk of recurrence comprises a subset of at least 10, 11, 12, 13, 14, 15, 16, 17, or 18 genes of the 34-gene panel. In some embodiments, the panel comprises a subset of at least 19, 20, 21, 22, 23, or 24 genes of the 34-gene panel. In some embodiments, the gene panel comprises a subset of at least 25, 26, 27, 28, 29, or 30 genes of the 34-gene panel. In some embodiments, the gene panel comprises a subset of 31, 32, or 33 genes of the 34-gene panel. In typical embodiments, the gene panel comprises all of the genes of the 34-gene panel.

The gene signature panel described herein is particularly useful in the methods of the present disclosure for determining risk of recurrence for personalized therapeutic management by selecting therapy, e.g., radiation therapy or repeat surgery for residual tumor for those patients who are determined to have a high risk of recurrence. The gene signature panel can also be useful for selecting chemotherapy and/or molecular therapies.

In a further aspect, the disclosure provides a method of processing a meningioma sample from a patient, the method comprising a meningioma sample from a patient; and quantifying levels of RNA expressed by the 36-gene signature panel, or a subset thereof as described herein; or quantifying level of RNA expressed by the 34-gene signature panel, or a subset thereof as described herein, compared to a reference score or a reference score scale obtained from analysis of meningioma tumors in patients that have meningioma. In some embodiments, the step of quantifying the level of RNA comprises performing an amplification reaction. In some embodiments, the amplification reaction is an RT-PCR reaction. In some embodiments, the step of quantifying the level of RNA comprises sequencing.

In a further aspect, the disclosure provides a method of processing a meningioma sample from a patient, the method comprising a meningioma sample from a patient; and quantifying levels of protein encoded by the 36-gene signature panel, or a subset thereof as described herein; or quantifying levels of protein encoded by the 34-gene signature panel, or a subset thereof as described herein, compared to reference levels of the proteins in control subjects. In some embodiments, the step of quantifying the level of protein comprises an immunoassay.

### Methods of Quantifying RNA Expression

In some embodiment, the methods of the present disclosure comprise detecting the level of RNA expression, e.g., mRNA expression, of a panel of 36 genes, or a subset thereof as described herein, in a tumor sample from a meningioma patient.

In some embodiment, the methods of the present disclosure comprise detecting the level of RNA expression, e.g., mRNA expression, of a panel of 34 genes, or a subset thereof as described herein, in a tumor sample from a meningioma patient.

The tumor sample can be any biological sample comprising meningioma cells. In some embodiments, the tumor sample is a fresh or archived sample obtained from the meningioma, e.g., during tumor resection. The sample also can be any biological fluid containing meningioma cells.

The level of RNA (e.g., mRNA) expression of the 36 genes of the signature panel as described above, or a subset thereof as described herein; or of the 34 genes of the signature panel as described above, or a subset thereof as described herein; can be detected or measured by a variety of methods including, but not limited to, an amplification assay, a hybridization assay, a sequencing assay, or an array. Non-limiting examples of such methods include quantitative RT-PCR, quantitative real-time PCR (qRT-PCR), digital PCR, nanostring technologies, serial analysis of gene expression (SAGE), and microarray analysis; ligation chain reaction, in situ hybridization, dot blot or northern hybridization; oligonucleotide elongation assays, mass spectroscopy, multiplexed hybridization-based assays, cDNA-mediated annealing, selection, extension, and ligation; mass spectrometry, and the like. In some embodiments, expression level is determined by sequencing, e.g., using massively parallel sequencing methodologies. For example, RNA-Seq can be employed to determine RNA expression levels.

In some embodiments, microarrays, e.g., are employed to assess RNA expression levels. The term “microarray” refers to an ordered arrangement of hybridizable probes, e.g., gene-specific oligonucleotides, attached to a substrate. Hybridization of nucleic acids from a sample to be evaluated is determined and converted to a quantitative value representing relative gene expression levels.

A pattern associated with increased risk of meningioma recurrence can include normalized expression levels in which some genes in the panel exhibit increased RNA expression levels, relative to normal controls and/or low-risk meningiomas; and other genes may exhibit decreased expression RNA expression levels relative to a normal control and/or low-risk meningioma. Thus, for example, increased expression of a gene, such as FOXM1, BIRC5, TOP2A, CDC2CDK1, SFRP4, and/or or MYBL2 may be associated with a higher risk in conjunction with decreased expression of BMP4, CTGF/CCN2, GAS1, PGR, and/or TMEM30B.

In some embodiments, the methods further comprise detecting level of RNA expression of one or more reference genes that can be used as controls to normalize expression levels. Such genes are housekeeping genes or otherwise typically expressed constitutively at a high level and can act as a reference for determining accurate gene expression level estimates. Examples of control genes include, but are not limited to, ARPC2, ATF4, ATP5B, B2M, CDH4, CELF1, CLTA, CLTC, COPB1, CTBP1, CYC1, CYFIP1, DAZAP2, DHX15, DIMT1, EEF1A1, FLOT2, GAPDH, GUSB, HADHA, HDLBP, HMBS, HNRNPC, HPRT1, HSP90AB1, MTCH1, MYL12B, NACA, NDUFB8, PGK1, PPIA, PPIB, PTBP1, RPL13A, RPLP0, RPS13, RPS23, RPS3, S100A6, SDHA, SEC31A, SET, SF3B1, SFRS3, SNRNP200, STARD7, SUMO1, TBP, TFRC, TMBIM6, TPT1, TRA2B, TUBA1C, UBB, UBC, UBE2D2, UBE2D3, VAMP3, XPO1, YTHDC1, YWHAZ, and 18S rRNA genes. Accordingly, a determination of RNA expression levels of the genes of interest, e.g., the gene expression levels of the panel of 36 genes as described herein, or a subset thereof; or the gene expression levels of the panel of 34 genes, or a subset thereof as described herein, may also comprise determining expression levels of one or more reference genes. Additional examples of control genes, e.g., for use with a 34 gene-panel, or subset thereof, are provided in Table 6. Accordingly, a determination of RNA expression levels of the genes of interest, e.g., the gene expression levels of the panel of 34 genes, or a subset thereof as described herein, may also comprise determining expression levels of one or more reference genes, such as those listed in Table 6.

The level of mRNA expression of each of the genes can be normalized to a reference level for one or more of the control genes. Alternatively, all of the assayed RNA transcripts or expression products, or a subset thereof, may also serve as reference. In some embodiments, the normalized amount of RNA may be compared to the amount found in a meningioma tumor reference set. A control value can be predetermined, determined concurrently, or determined after a sample is obtained from the subject. Thus, for example, the reference control level for normalization can be evaluated in the same assay or can be a known control from a previous assay.

### Methods of Quantifying Protein Levels

In some embodiments, methods of determining expression levels of the 36 genes in the signature panel described herein, or a subset of the 36 genes as described above can comprise determining the level of the polypeptides encoded by the genes in the panel, or subset thereof, in the tumor tissue.

In some embodiments, expression is determined by assess the level of proteins encoded by genes in the 36-gene panel, or a subset of the 36-gene panel as described herein; or levels of proteins encoded by genes in the 34-gene panel, or a subset of the 34-gene panel as described herein. Thus, for example, expression may be assessed using an immunoassay, such as a sandwich immunoassay, competitive immunoassay, and the like. In some embodiments, protein expression may be determined using mass spectrometry methods or by electrophoretic methods. In some embodiments, expression of polypeptides encoded by genes in the panel can be detected simultaneously using a multiplex assay, such as a multiplex ELISA. In other embodiments, protein expression can be determined using

The level of protein encoded by each of the genes in the 36-gene panel, or the 34-gene panel, or a subset of the 36-gene panel or the 34-gene panel as described in the present application, can be normalized to a reference level of protein encoded by one or more of the control genes. Alternatively, all of the assayed protein expression products, or a subset thereof, may also serve as reference. In some embodiments, the normalized amount of protein for each gene may be compared to the amount found in a meningioma tumor reference set. A control value can be predetermined, determined concurrently, or determined after a sample is obtained from the subject. Thus, for example, the reference control level for normalization can be evaluated in the same assay or can be a known control from a previous assay.

### Establishing Meningioma Recurrence Risk Scores

After determining the normalized expression of level of the 36-gene signature panel, or the 34-gene signature, or a subset of the 36-gene or 34-gene panel as described herein, the method presented herein includes calculating a risk score, e.g., a risk score based on the level of RNA expression of each member of the gene panel. The level of expression of the 36 genes or the 34 genes, or a subset of the 36-gene or 34-gene panel as described herein, can be equally weighted in the risk score. In some embodiments, the level of expression of each gene is weighted with a predefined coefficient. The predefined coefficient can be the same or different for the genes and can be determined by a statistical or machine learning algorithm such as linear regression, ridge or lasso regression, elastic net regression, regularized Cox regression, support vector machine, and the like.

In some embodiments, the risk score is generated to provide a tumor-specific gene signature risk score between 0 and 1 based on a machine learning classifier, e.g., the elastic net regression classifier as illustrated in the Examples section, or another method such as linear regression, ridge or lasso regression, regularized Cox regression, support vector machine, naïve Bayes classification, and the like.

One of ordinary skill in the art recognizes that a variety of statistical methods can be used for comparing the expression level of the genes. In some embodiments, a patient's risk score is categorized as “high,” “intermediate,” or “low” relative to a reference scale, e.g., a range of risk scores from a population of reference subjects that have the same cancer as the patient. In some cases, a high score corresponds to a numerical value in the top tertile (e.g., the highest ⅓) of the reference scale; an intermediate score corresponds to the intermediate tertile (e.g., the middle ⅓) of the reference scale; and a low score corresponds to the bottom tertile (e.g., the lowest ⅓) of the reference scale. In other embodiments, a high score represents a risk score that is 0.66 or above, e.g., 0.66, 0.67, 0.70, 0.75, 0.80, 0.85, 0.90, 0.95, 0.99 or 1.0 based on a normalized, standardized reference scale on a scale of 0 to 1. In other embodiments, a patient's risk score is compared to one or more threshold value(s) to provide a likelihood of recurrence of the meningioma. In some cases, the high risk score corresponds to a numerical value, e.g., a risk score in the top 5%, top 10%, top 15%, top 20%, top 25%, top 30%, top 35%, top 40%, top 45%, top 50%, or top 60% of the reference scale. In some cases, the high risk score corresponds to a numerical value, e.g., a risk score in the top 5%, top 10%, top 15%, top 20%, top 25%, top 30%, top 35%, top 40%, top 45%, or top 50% of the reference scale. In some cases, the high risk score corresponds to a numerical value, e.g., a risk score in the top 5%, top 10%, top 15%, top 20%, top 25%, top 30%, top 35%, or top 40% of the reference scale.

In order to establish a reference risk scale or a threshold value for practicing the method of this invention, a reference population of subjects can be used. In some embodiments, the reference population may have the type of cancer or tumor as the test patient, but may represent a range of subtypes of stages of the cancer. In some embodiments, the reference populations may have the same subtype and/or stage of cancer or tumor as the test patient. The subjects in the reference population can be within the appropriate parameters, if applicable, for the purpose of screening for and/or monitoring cancer using the methods provided herein. In some embodiments the reference scale is a plurality of risk scores derived from analysis of meningioma tumors from a population of reference patients. In some embodiments, the reference population may take into account various characteristics, such as WHO Grade, extent of resection, prior treatment status, prior radiation status, NF2 status, tumor size, multifocal nature of the tumor, presence of brain invasion, and/or Ki67 labeling index. Optionally, the reference subjects are of same gender, similar age, or similar ethnic background.

### Computer-Implemented Methods, Systems, and Devices

Any of the methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps. Although presented as numbered steps, steps of methods herein can be performed at a same time or in a different order. Additionally, portions of these steps may be used with portions of other steps from other methods. Also, all or portions of a step may be optional. Any of the steps of any of the methods can be performed with modules, circuits, or other means for performing these steps.

Any of the computer systems mentioned herein may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. For example, in some embodiments, a computer system may include storage device(s), a monitor coupled to a display adapter, and a keyboard. Peripherals and input/output (I/O) devices, which couple to an I/O controller, can be connected to the computer system by any number of means known in the art, such as a serial port. For example, a serial port or external interface (e.g. Ethernet, Wi-Fi, etc.) can be used to connect a computer system to a wide area network such as the Internet, a mouse input device, or a scanner. The interconnection via a system bus allows the central processor to communicate with each subsystem and to control the execution of instructions from system memory or the storage device(s) (e.g., a fixed disk, such as a hard drive or optical disk), as well as the exchange of information between subsystems. The system memory and/or the storage device(s) may embody a computer readable medium. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present disclosure can be implemented in the form of control logic using hardware (e.g., an application specific integrated circuit or field programmable gate array) and/or using computer software with a generally programmable processor in a modular or integrated manner. As user herein, a processor includes a multi-core processor on a same integrated chip, or multiple processing units on a single circuit board or networked. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

### Kits

The present disclosure also provides kits for practicing the methods described herein. The kits may comprise any or all of the reagents to perform the methods described herein. In some embodiments a kit may include any or all of the following: assay reagents, buffers, probes that target each member of the 36-gene panel, or a subset as described herein; or that target at least one of the members of the 34-gene panel, or subset as described herein, such as hybridization probes and/or primers, antibodies or other moieties that specifically bind to at least one of the polypeptides encoded by the genes described herein, etc. In addition, the kit may include reagents such as nucleic acids, hybridization probes, primers, antibodies and the like that specifically bind to a reference gene or a reference polypeptide. The kit may comprise probes to one or more reference genes identified herein, such as, ARPC2, ATF4, ATP5B, B2M, CDH4, CELF1, CLTA, CLTC, COPB1, CTBP1, CYC1, CYFIP1, DAZAP2, DHX15, DIMT1, EEF1A1, FLOT2, CAPDH, GUSB, HADHA, HDLBP, HMBS, HNRNPC, HPRT1, HSP90AB1, MTCH1, MYL12B, NACA, NDUFB8, PGK1, PPIA, PPIB, PTBP1, RPL13A, RPLP0, RPS13, RPS23, RPS3, S100A6, SDHA, SEC31A, SET, SF3B1, SFRS3, SNRNP200, STARD7, SUMO1, TBP, TFRC, TMBIM6, TPT1, TRA2B, TUBA1C, UBB, UBC, UBE2D2, UBE2D3, VAMP3, XPO1, YTHDC1, YWHAZ, and 18S rRNA; and/or one of the reference genes listed in Tables 4 and 6.

The term “kit” as used herein in the context of detection reagents, are intended to refer to such things as combinations of multiple gene expression product detection reagents, or one or more gene expression product detection reagents in combination with one or more other types of elements or components (e.g., other types of biochemical reagents, containers, packages such as packaging intended for commercial sale, substrates to which gene expression detection product reagents are attached, electronic hardware components, etc.).

In some embodiments, the present disclosure provides oligonucleotide probes attached to a solid support, such as an array slide or chip. Construction of such devices are well known in the art.

A microarray can be composed of a large number of unique, single-stranded polynucleotides, usually either synthetic antisense polynucleotides or fragments of cDNAs, fixed to a solid support. In typical embodiments, a microarray of the present invention comprises probes that target expression of no more than 1,000 genes, nor more than 500 genes, nor more than 200 genes or no more than 100 genes, including the 36-gene panel described herein, or a subset of the panel as described herein; or including the 34-gene panel described herein, or a subset of the panel as describe herein. Typical polynucleotides are preferably about 6-60 nucleotides in length, more preferably about 15-30 nucleotides in length, and most preferably about 18-25 nucleotides in length. For certain types of arrays or other detection kits/systems, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length.

In addition, the kits may include instructional materials containing directions (i.e., protocols) for the practice of the methods provided herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### I. EXAMPLES

The following examples are offered to illustrate, but not to limit, the claimed invention. The Examples describe the identification and validation of a panel of genes for assessing risk of recurrence of meningioma.

### Example 1. Identification of a First Panel of Genes for Assessing Meningioma

### Methods

### Discovery and Validation Patient Cohorts

The discovery cohort of patients with meningioma that were treated with resection was from cases between 1990 and 2015 from the University of California San Francisco (UCSF). Patients were retrospectively identified from an institutional clinical database and cross-referenced with samples in the UCSF Brain Tumor Center Pathology Core and Tissue Biorepository. Meningiomas of sufficient quantity and quality for molecular analysis that were associated with patients who had sufficient clinical data, including pathology reports, surgical reports, pre-operative and surveillance brain imaging. For all cases, pathologic re-grading was undertaken based on the most recent WHO histopathologic criteria⁶, and diagnostic imaging was re-reviewed to confirm the extent of resection and determine the occurrence and timing of local recurrence, which was defined as local recurrence of any size after gross-total resection (GTR), or growth of ≥20% along any dimension after subtotal resection (STR). Mortality data and cause of death were extracted from the electronic medical record, institutional cancer registry, Surveillance, Epidemiology, and End Results (SEER), Department of Motor Vehicles (DMV), Social Security, and nationwide hospital databases, and publicly available obituaries. This study was approved by the Institutional Review Board, Human Research Protection Program Committee on Human Research, protocol 10-03204.

In order to identify an independent validation dataset of patients with meningiomas that were treated with resection, a search was undertaken of the Gene Expression Omnibus (GEO) repository using the term “meningioma”, filtered for “expression profiling by array” and human samples, resulting in 37 results. Each result was manually evaluated, and a total of 17 GEO entries (GEO Accession numbers: GSE4039, GSE4780, GSE9438, GSE12530, GSE16581, GSE16153, GSE16156, GSE8557, GSE32197, GSE58037, GSE43290, GSE88720, GSE85135, GSE84263, GSE77259, GSE74385, GSE54934) representing 13 unique datasets of microarray gene expression data of meningioma tumor samples were identified. Next, datasets were screened for public availability of clinical endpoints matched to tumor samples, including, at a minimum, WHO grade, time to local recurrence or censorship and recurrence status, and time to death or censorship and vital status. Only one dataset fit these criteria (GSE58037), comprising 68 tumor samples from 68 unique patients with whole genome expression data using the Affymetrix U133 Plus 2.0 array, of which 56 had complete clinical data³².

### Targeted Gene Expression Analysis and Immunohistochemistry

As previously described²³, total RNA was extracted from tumor cores from formalin-fixed paraffin-embedded (FFPE) tissue blocks containing 75% or more tumor cells as determined by hematoxylin and eosin (H&E) staining. Concentrations were determined based on spectrophotometry and RNA integrity assessed using a bioanalyzer (Agilent, San Francisco, Calif. The GX Human Cancer Reference Nanostring panel codeset, with 30 additional meningioma related genes (266 total gene probes, Table 2, were synthesized by NanoString technologies (Seattle, Wash.). RNA (200 ng per meningioma) was analyzed with the NanoString nCounter Analysis System at NanoString Technologies, according to the manufacturer's protocol. Immunohistochemistry (IHC) was performed on previously generated formalin-fixed paraffin embedded tissue microarrays containing 1mm or 2mm cores in duplicate or in triplicate. Five-micron sections were stained using standard techniques on a Roche Ventana BenchMark XT automated immunostainer (Roche Diagnostics, Indianapolis, Ind.) using a rabbit monoclonal primary SFRP4 antibody (clone EPR9389, 1:1000 dilution, Abcam, Cambridge, Mass.), or a rabbit polyclonal primary TMEM30B antibody (clone EPR14409, 1:4000 dilution, Abcam), with polymeric secondary detection system (ultraView, Ventana). Stains were scored as “low” when the stains are negative or weak positive, and as “high” when the stains are moderate to strong positive.

### Bioinformatic and Statistical Analyses

In order to train a gene expression classifier able to discriminate meningiomas with poor clinical outcomes, defined as a faster time to local recurrence, cases were dichotomized based on time to local recurrence rather than on recurrence status, as a meningioma that recurred many years after resection may not necessarily represent a more aggressive meningioma than one which did not recur, but was lost to follow up shortly after surgery. Thus, recurrent cases were dichotomized into poor- and baseline-outcome classes based on time to recurrence falling below or above the median time to local recurrence.

NanoString data were pre-processed according to manufacturer guidelines. Background thresholding was performed utilizing a threshold of 2 standard deviations above the mean of built in negative controls. Next, log₂-transformed count data were centered and scaled within-meningiomas using a Z-score transformation. The method of shrunken centroids, also known as prediction analysis for microarrays (PAM), is an extension of the nearest centroid classifier and linear discriminant analysis³³, and was used to identify a subset of genes from the discovery cohort that were associated with poor outcomes (pamr: Pam: Prediction Analysis for Microarrays. R package version 1.56.1)³⁴. K-fold cross validation was performed using the pamr.cv function to determine the optimal shrinkage threshold. Importantly, PAM has been widely used to generate classifiers and gene signatures based on gene expression microarray data³⁵⁻³⁷.

In order to generate a generalizable risk score based on the genes of interest identified by PAM, Z- and loge-transformed counts of genes of interest were further scaled and constrained using the softmax transformation³⁸, also known as the normalized exponential function, such that the sum of values of each gene of interest within a given meningioma equaled 1.

Next, an elastic net regression classifier was trained utilizing K-fold cross-validation, and using the above transformed values as input and the probability of classification as poor-outcome as output. The probability of poor-outcome between 0 and 1 was defined as the meningioma gene signature risk score. Elastic net regression was performed using the ElasticNetCV function of the Scikit-learn package in Python³⁹.

Microarray data from the validation cohort were pre-processed as described previously⁴⁰. In brief, raw probe intensity values in .CEL format were normalized using the robust multichip average (RMA) method with default settings in the Bioconductor package in R⁴¹. Next, we applied an identical set of transformations to the data, including loge transformation followed by intrasample Z-score centering and softmax scaling and constraining. Finally, the elastic net classifier from above was applied to the genes of interest of the validation cohort to obtain gene signature risk scores.

CNV data was also obtained from the validation cohort, as previously described⁴⁰. In brief, copy number calls were generated based on the Affymetrix GeneChip Human Mapping 100K single nucleotide polymorphism array, and using the Affymetrix GTYPE CNAT (v3.0) algorithm using default parameters.

Gene set enrichment analysis was performed using ConsensusPathDB⁴², and protein-protein interaction analysis, clustering, and visualization was performed with the STRING database⁴³. All other statistical analyses including Cox proportional hazards regression, Kaplan Meier survival analysis and log-rank tests, and other standard statistical tests were performed in JMP (JMP®, Version 14.0. SAS Institute Inc., Cary, N.C., 1989-2019).

### Results

The characteristics of the discovery and validation cohorts are summarized in Table 1.

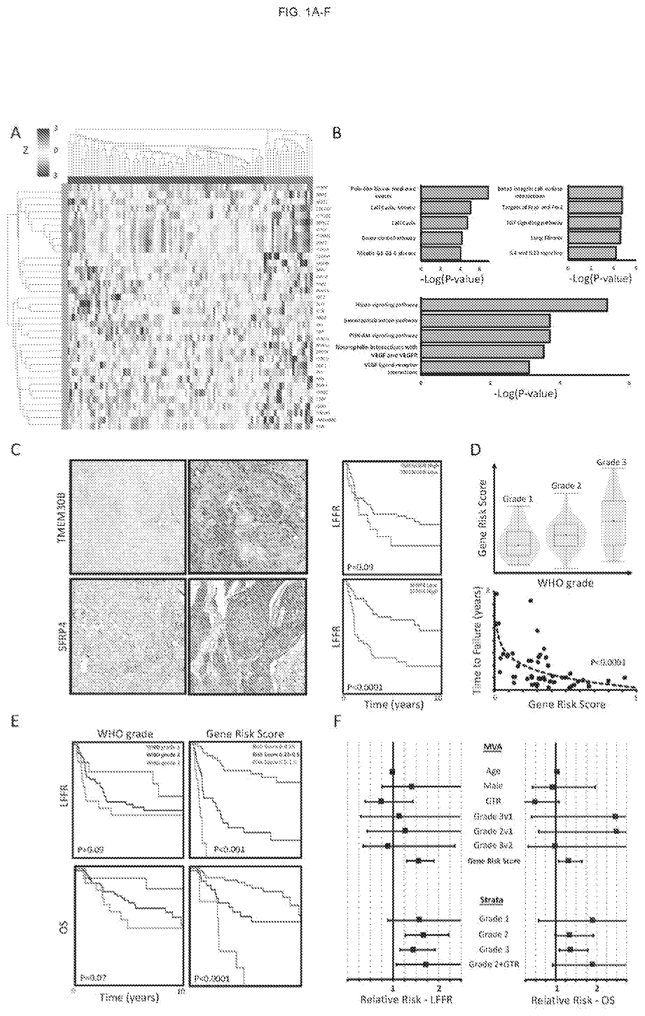

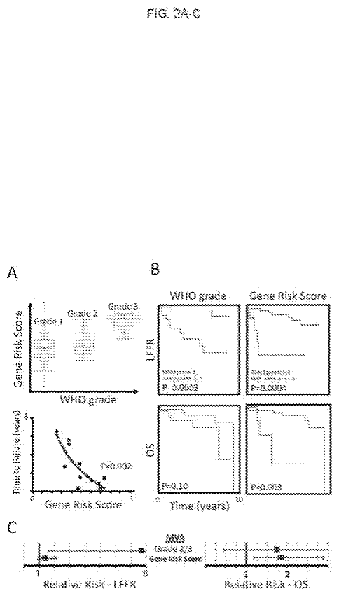

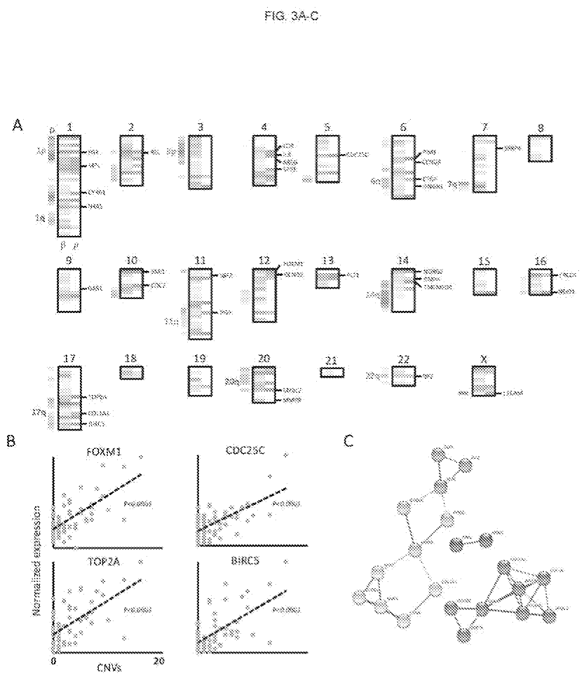

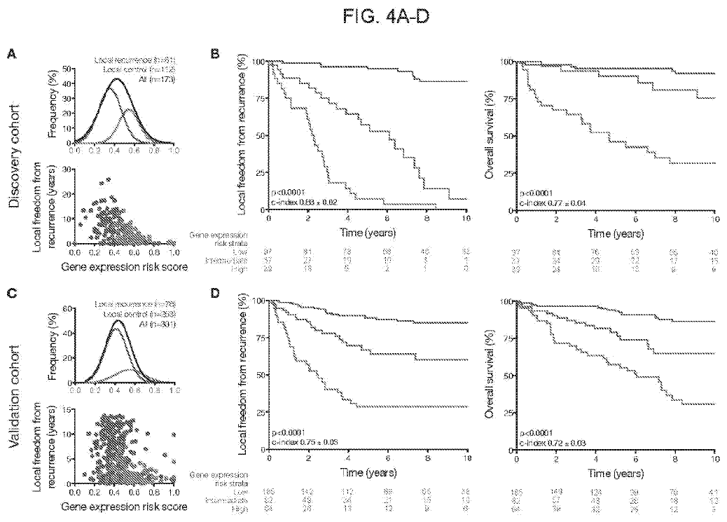

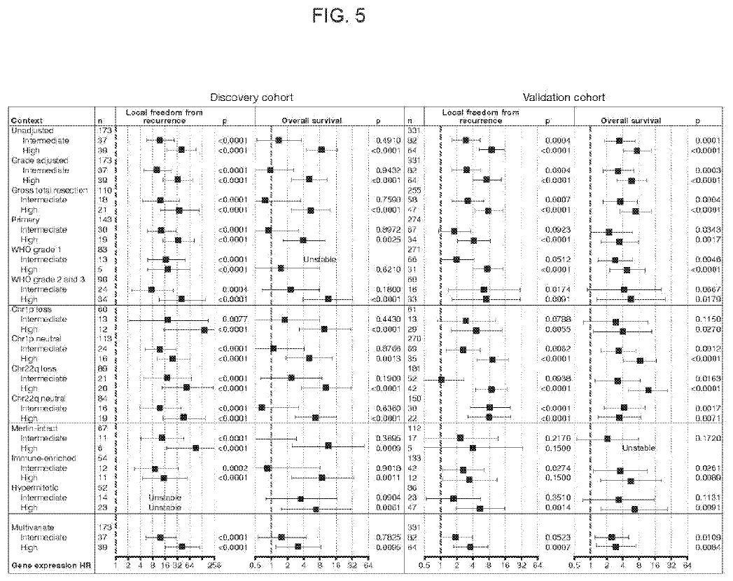

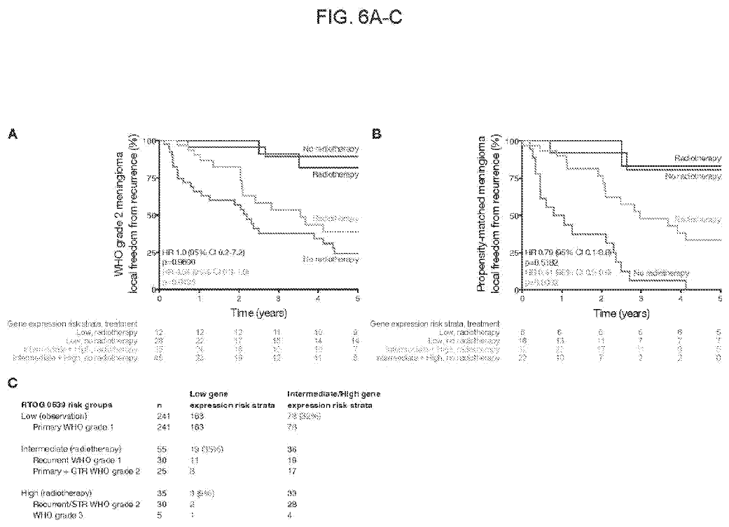

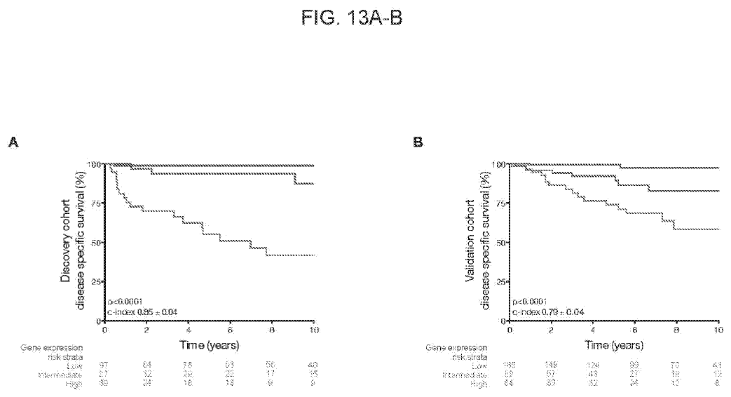

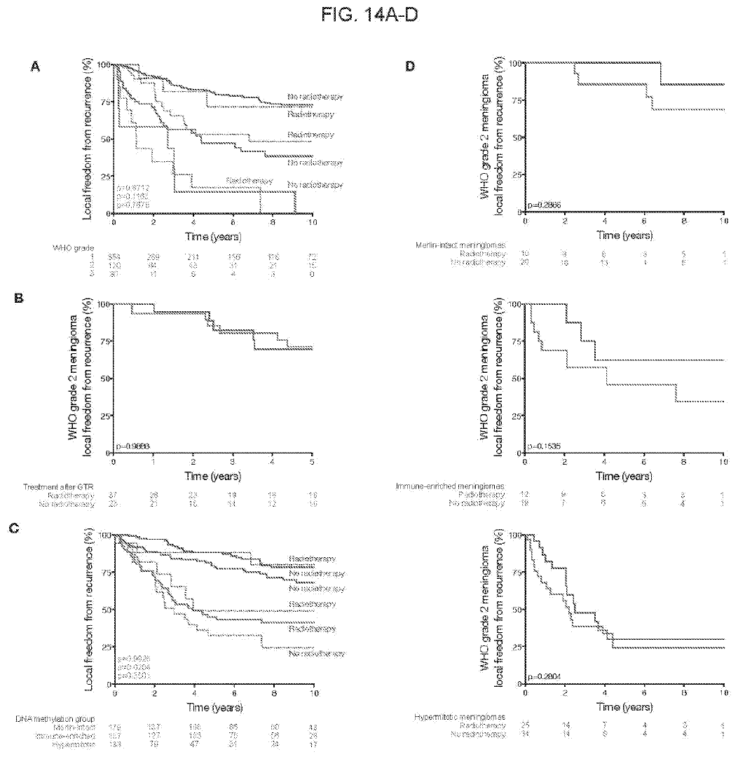

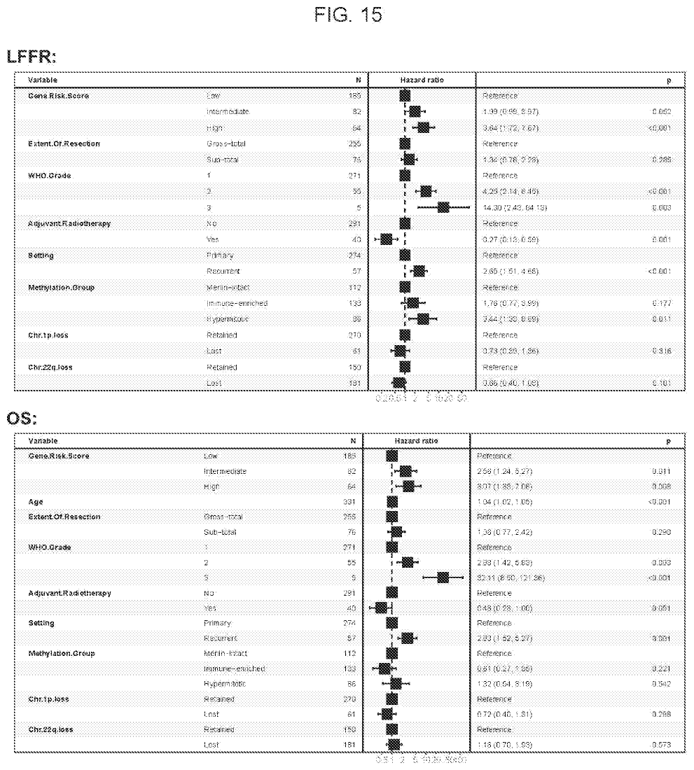

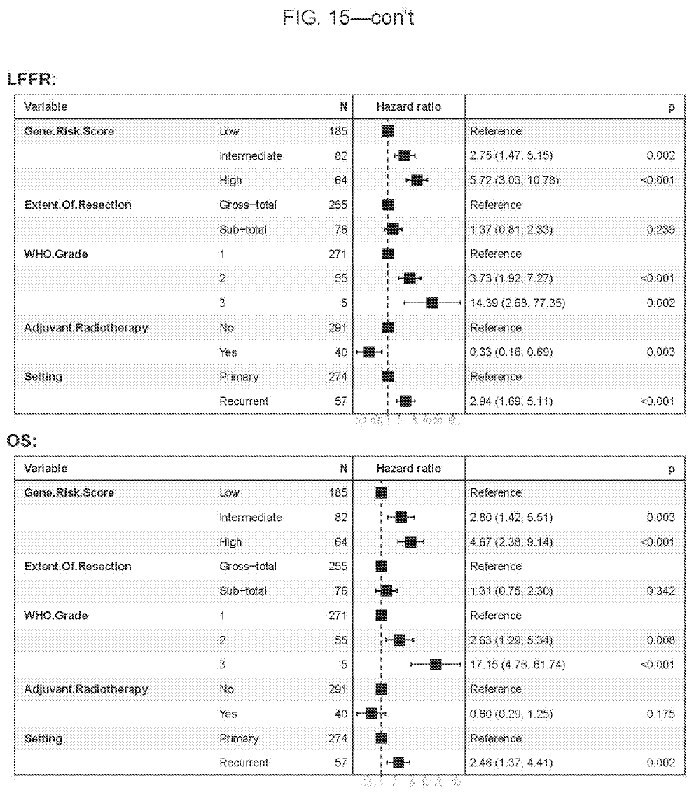

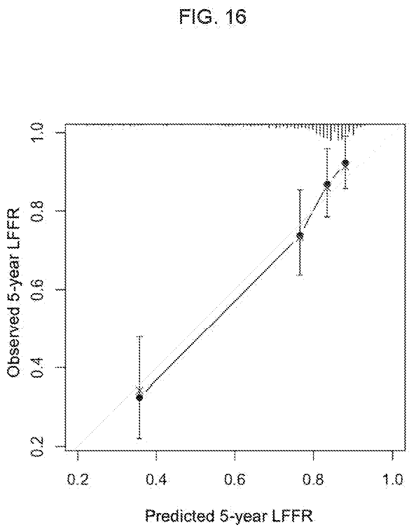

After dichotomizing the discovery cohort into poor-outcome (N=25, median local freedom from recurrence [LFFR] 0.70 years, median OS 2.5 years) and baseline-outcome cases (N=71, median LFFR not reached, median OS 11.9 years), the method of shrunken centroids identified a set of 36 genes that distinguished between outcome subgroups (FIG. 1A). In order to confirm the prognostic significance of the genes comprising our meningioma gene score, unsupervised hierarchical clustering was performed on cases from the discovery cohort based on expression of genes of interest (FIG. 1A), which demonstrated robust clustering of cases into 2 subgroups with significant differences in LFFR (median 0.92 vs 7.8 years, P<0.0001, Log-rank test) and OS (4.0 years vs 14.4 years, P=0.0003, Log-rank test). The subgroup of meningioma cases with the worst outcomes showed increased expression of genes associated with cell cycle regulation and mitosis (FIG. 1B, FIG. 3C), including FOXM1⁴⁴, BIRC5⁴⁵, TOP2A⁴⁶, CDC2⁴⁷, SFRP4⁴⁸, and MYBL2⁴⁹, as well as concomitant decreased expression of BMP4, a signaling molecule involved in embryonic development, stem cell differentiation, and bone and cartilage morphogenesis⁵⁰; CTGF, which is important for wound healing and fibrosis⁵¹; GAS1, a tumor suppressor⁵²; progesterone receptor (PGR), which has been implicated in low grade meningiomas⁵³; and TMEM30B, a transmembrane gene product with unknown function^{54,55}. IHC for representative enriched or suppressed gene products from each cluster confirmed that cases with high SFRP4 staining had significantly worse LFFR (FIG. 1C, p<0.0001), while cases with low or absent TMEM30B staining showed a trend towards worse LFFR (FIG. 1C, P=0.09). In further support of the prognostic value of these genes for meningioma outcomes, we have previously shown increased IHC staining of FOXM1 to be strongly associated with worse LFFR and OS²³. Closer interrogation of the gene signature revealed that multiple prognostic genes were contained at chromosomal loci frequently affected by CNVs in high grade meningiomas²², including 1p, 1q, 6q, 17q, and 20q (FIG. 3A). Consistently, the expression of 4 genes that were enriched in meningiomas with poor outcomes, FOXM1, TOP2A, BIRC5 and CDC25C, was positively associated with the number of CNVs in cases from the validation cohort (FIG. 3B). These data suggest our prognostic gene expression signature for meningioma recurrence after resection captures genes that are recurrently altered through CNVs as meningiomas dedifferentiate from indolent to clinically aggressive cancers^{22,23}.

Next, we utilized the 36-gene signature of poor meningioma outcomes to generate a tumor specific gene signature risk score between 0 and 1 based on an elastic net regression classifier that achieved a cross-validation accuracy of 0.80 and AUC of 0.86 in distinguishing poor- and baseline-outcome cases in the discovery cohort. The meningioma gene signature risk score based on this classifier achieved a concordance index (c-index) of 0.75±0.03 (P<0.0001, Wald test) for LFFR, and 0.72±0.04 for OS (P<0.0001, Wald test), within the discovery cohort. The risk score was only weakly correlated with WHO grade (FIG. 1D), yet was strongly correlated with faster time to failure (F-test, P<0.0001, FIG. 1D), and significantly outperformed WHO grade in stratifying cases by LFFR and OS (FIG. 1E). In order to investigate the clinical utility of the meningioma gene signature risk score, we constructed a multivariate Cox model of LFFR and OS, incorporating age, sex, extent of resection, WHO grade, and meningioma gene signature risk score (FIG. 1D). After adjusting for these clinical covariates, a higher meningioma gene signature risk score remained significantly associated with worse LFFR (FIG. 1F, relative risk [RR] 1.56 per 0.1 gene signature risk score increase, 95% confidence interval [CI] 1.30-1.90, P<0.0001) and OS (RR 1.32 per 0.1 increase, 95% CI 1.07-1.64, P=0.01). Similarly, after stratifying cases in the discovery cohort by WHO grade, the meningioma gene signature risk score remained significantly associated with worse LFFR among WHO grade II (RR 1.67 per 0.1 increase, 95% CI 1.27-2.22, P=0.0003) and III (RR 1.45 per 0.1 increase, 95% CI 1.15-1.92, P=0.003) tumors on univariate analysis, and trended towards significance among WHO grade I tumors (P=0.10), likely owing to the small sample size of grade I tumors in the discovery cohort. The meningioma gene signature risk score was similarly associated with worse LFFR among the subgroup of atypical WHO grade II meningiomas status post GTR (N=26, RR 1.72 per 0.1 increase, 95% CI 1.08-2.86, P=0.03), and remained significantly associated with worse LFFR among primary meningiomas without prior radiation (N=60, RR 2.0 per 0.1 increase, 95% CI 1.44-2.81, P<0.0001) with a trend towards worse OS (RR 1.50 per 0.1 increase, 95% CI 0.98-2.35, P=0.06) in this subgroup (FIG. 1F).

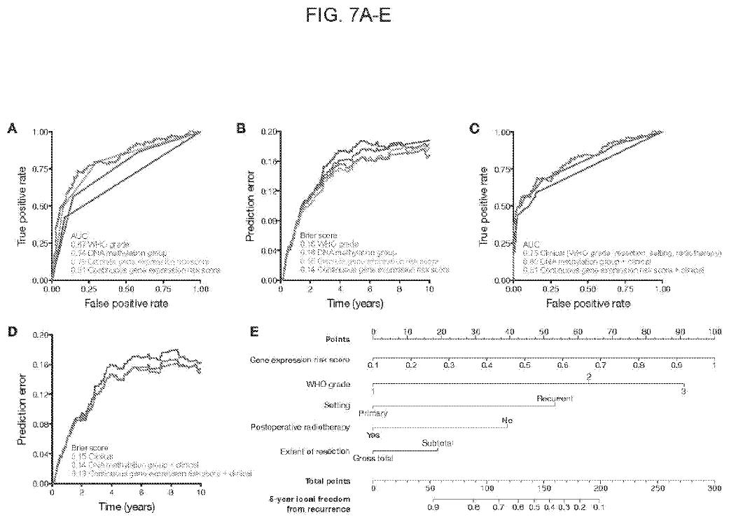

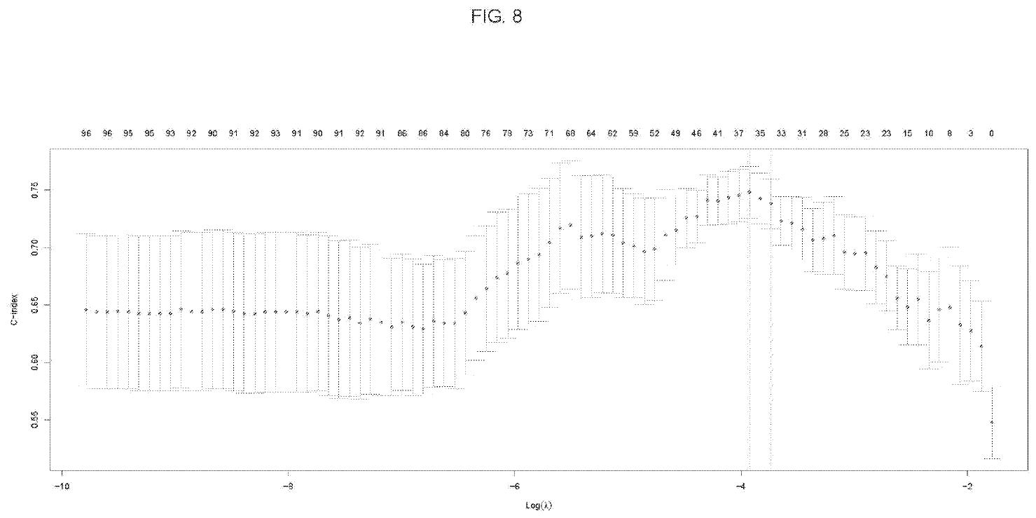

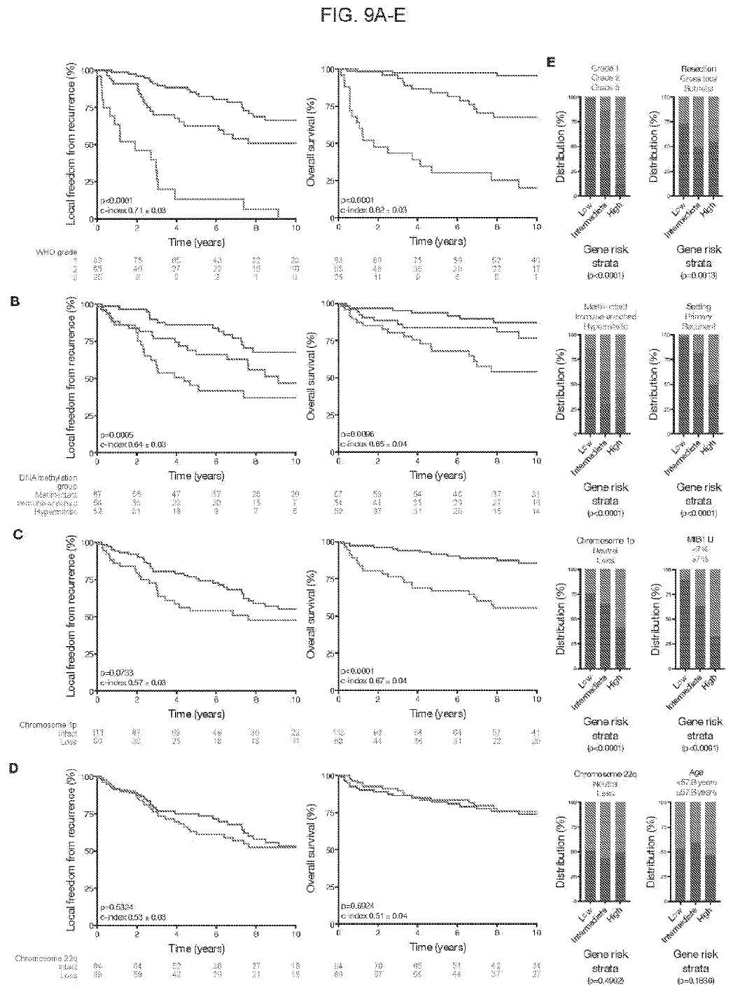

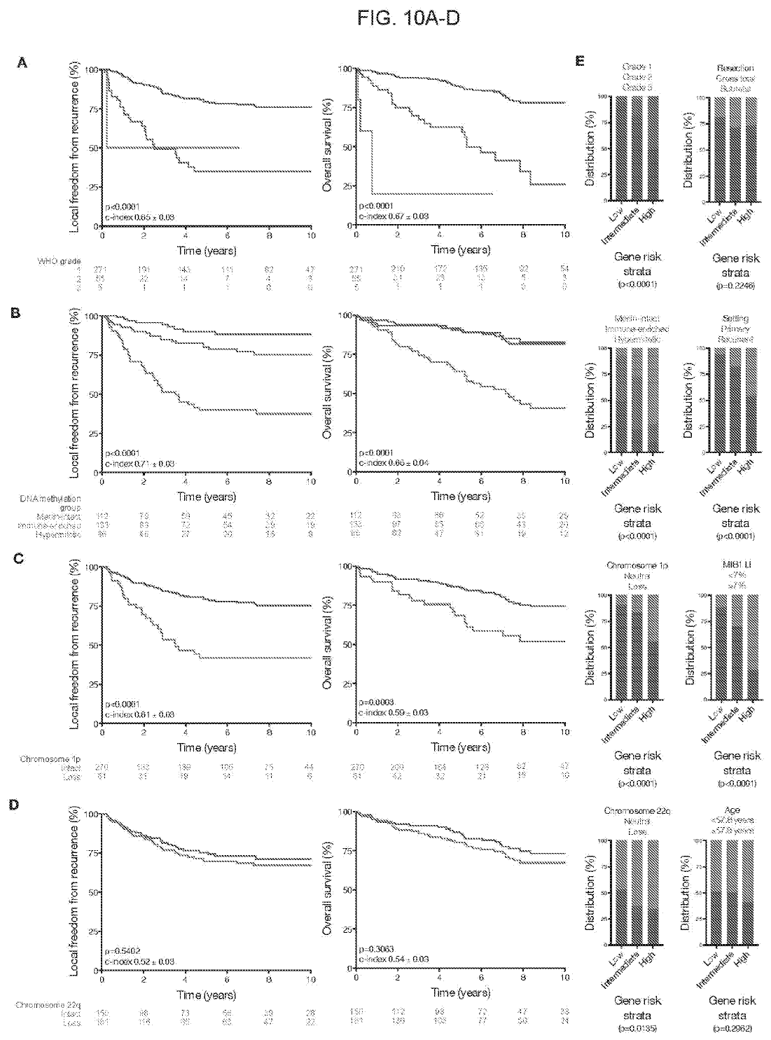

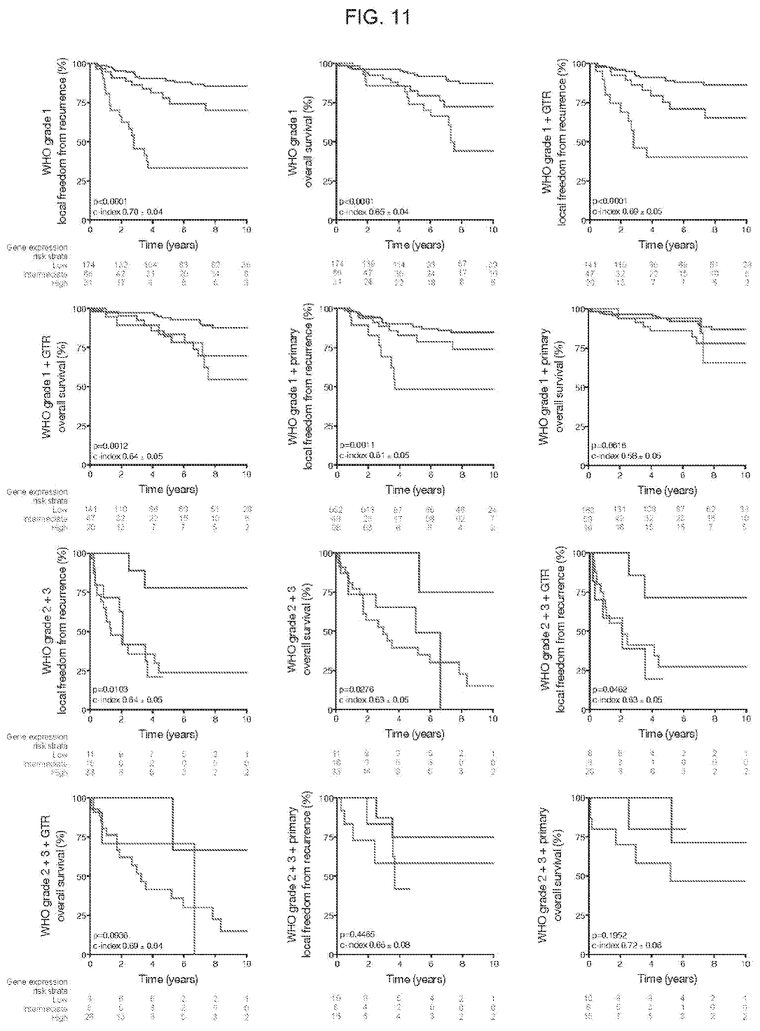

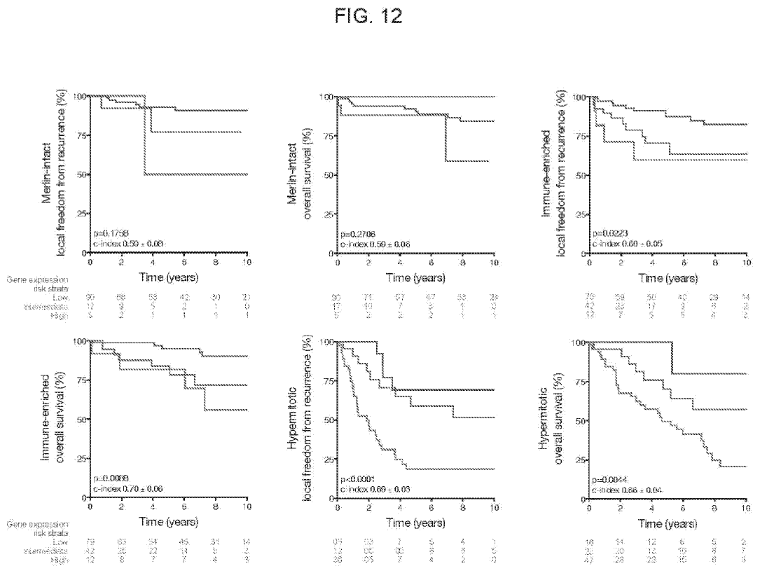

Finally, we sought to validate the prognostic utility of our meningioma gene signature risk score in an independent cohort of meningiomas status post resection at an independent institution. The validation cohort we identified was more representative of a general population of patients with meningiomas, with fewer events of local recurrence (20% vs 58%, Table 1) or mortality (18 vs 42%). Nevertheless, the meningioma gene signature risk score was again associated with WHO grade and strongly correlated with faster time to failure (F-test, p=0.002, FIG. 2A). Moreover, our meningioma gene signature risk score was able to accurately stratify cases by LFFR (FIG. 2B, p=0.0004, Log-rank test), significantly outperformed WHO grade in stratifying cases by OS (P=0.003 vs P=0.10, Log-rank test), and achieved a c-index of 0.76±0.07 (P=0.01, Wald test) for LFFR, and 0.76±0.11 for OS (P=0.002, Wald test). Finally, after adjusting for WHO grade, a higher meningioma gene signature risk score remained significantly associated with worse OS (FIG. 2C, RR 1.86 per 0.1 increase, 95% CI 1.19-2.88, P=0.005) (FIG. 2C).

### Summary of Findings for Example 1

More than 15-20% of meningiomas are high grade, and in clinical practice a subset of patients with meningiomas of all grades experience a clinically aggressive course associated with significant morbidity and mortality⁵⁶⁻⁵⁹. In order to identify better prognostic markers to help delineate clinically aggressive meningiomas, we performed targeted gene expression analysis on a discovery cohort of meningioma cases that were enriched for clinical endpoints of local recurrence and disease specific mortality. We identified a 36-gene signature of clinically aggressive meningioma and derived a meningioma gene signature risk score between 0 and 1 that outperformed WHO grade in stratifying cases by risk of recurrence and survival. Moreover, we demonstrated the utility of this gene signature in risk stratifying meningioma patients from an independent validation cohort that is more representative of typical meningioma patients.

### Clinical Significance

Longitudinal studies of meningioma patients with long term follow up indicate that the 10-year recurrence rates after primary resection of benign, WHO grade I tumors are upwards of 20-30%⁵⁶⁻⁵⁸, and 40-50% for WHO grade II tumors⁶⁰⁻⁶⁵. These recurrences and subsequent therapies in the form of repeat craniotomy and ionizing radiation are causes of significant morbidity and, in many cases, mortality^{57,66,67}. Yet, due to the variable latency of many meningioma recurrences and the advanced age of most meningioma patients, it remains challenging to a priori identify patients at risk of recurrence and to appropriately tailor adjuvant management, which can include surveillance, radiotherapy, and re-resection in the event of subtotal primary surgery. Younger patients, in particular, may stand to gain the most from appropriate adjuvant management in preventing the morbidity and mortality associated with local recurrence, yet may also be more likely to experience the long-term toxicities of aggressive therapy, which can include cognitive or neurological effects due to radiation or repeat surgery^{68,69,} radiation necrosis, and the risk of secondary malignancies or malignant transformation due to radiation therapy^{68,70.} These challenges underline the urgent need for robust and clinically practical prognostic biomarkers for meningioma. To that end, the gene signature and risk score identified here could be used to identify high-risk patients who may benefit from aggressive adjuvant management, and conversely, to spare low-risk patients the potential toxicities of more aggressive interventions. Similar gene expression based assays have had a substantial impact on the care of patients with other common cancers, helping to guide the appropriate use of adjuvant chemotherapy among breast cancer patients²⁹, and helping inform the use of active surveillance among patients with prostate cancer³¹.

### A Gene Expression Signature of Clinically Aggressive Meningioma

The meningioma gene signature we report consists of enriched genes involved in cell cycle regulation, mitosis, and proliferation, and suppressed genes involved in stem cell differentiation, wound healing, and tumor suppressor functions³⁸⁻⁴⁹. As an added marker of external validity, many of the prognostic genes we identified have previously been implicated in clinically aggressive meningiomas, including FOXM1^{23,71-73}, TOP2A^{23,74}, BIRC5⁷⁴, MYBL2¹⁰ and CDC2⁷⁴. Prior work demonstrated that elevated expression of FOXM1 and FOXM1 target genes, including TOP2A, was associated with poorer outcome²³. BIRC5, whose gene product is also known as Survivin, is co-expressed with FOXM1 in breast cancer in patients with poor outcomes and drug-resistance⁷⁵. Similarly, FOXM1 and MYBL2 are associated with a subgroup of meningiomas identified by gene expression clustering to have poorer outcomes¹⁰. Thus, these components of our meningioma gene signature and risk score may be representative of a common or convergent set of genes associated with meningioma cell proliferation and mitosis, which are hallmarks of clinically aggressive cancers.

The meningioma gene signature we identified also contains a number of genes that are suppressed in meningiomas with poor outcomes. Indeed, many of these genes have previously been shown to be negatively correlated with poor meningioma outcomes. Loss of progesterone receptor staining on immunohistochemistry is associated with elevated proliferation indices, higher meningioma grade, and greater risk of recurrence⁷⁶. Similarly, NDRG2 is a tumor suppressor gene that is frequently inactivated among more aggressive meningiomas⁷⁷. Interestingly, a minor allele variant of ERCC4, a DNA repair gene, was associated with a significantly elevated risk of meningioma⁷⁸. Other notably underrepresented genes in poor-outcome meningiomas identified in our gene signature include BMP4, which has previously been shown to be suppressed in high grade meningiomas⁷⁹, as well as TMEM30B and CTGF, both of which were identified in a prior study as frequently suppressed among recurrent meningiomas, and associated with chromosomal 6q and 14q losses⁵⁴. Indeed, our analysis indicates that many genes selected by the gene signature reside at chromosomal locations frequently altered in higher grade meningioma. Furthermore, our investigation of genes correlated with chromosomal aberration in our validation cohort identified a tightly co-expressed network of proliferative genes including FOXM1, TOP2A, CDC25C, and BIRC5 to be highly linearly correlated with higher number of CNVs. Accumulation of CNVs is increasingly being understood to be a key hallmark of meningioma progression and a marker of aggressive tumors^{23,32}, and the genes highlighted by our gene signature may thus represent a core set of deregulated genes downstream of CNV accumulation which contribute to the increased proliferation, therapy resistance, and invasiveness of clinically aggressive meningioma.

Elements of the present study that distinguish it from previous investigations include: (i) the use of a discovery cohort significantly enriched for adverse clinical endpoints, including mortality, the majority of which were documented to be secondary to disease progression, which allowed for improved performance of bioinformatic algorithms to identify discriminatory genes; (ii) the choice to model poor-outcome based on time to recurrence rather than recurrence as a binary variable, which better captured the clinical behavior of cases; (iii) validation of our meningioma gene signature risk score using an independent cohort of meningiomas that were representative of the general population of meningioma patients; and (iv) integration of multiple genes whose altered expression have previously been described to be prognostic in meningioma into a unified prognostic model.

The present study also has several limitations. First, the study is retrospective and thus limited by the inherent biases of all retrospective investigations. We attempted to mitigate these biases by utilizing multiple data sources for collection of clinical endpoints, and by performing careful re-review of meningioma pathology and radiology. Second, both our discovery and validation cohorts represent cases from two academic institutions. While the validation cohort is more representative of a general population of meningioma patients, it nevertheless may not be representative of the larger clinical population encountered outside of academic institutions. Along these lines, our discovery cohort contained few WHO grade I tumors. With this limitation in mind, it is perhaps not surprising that our meningioma gene signature risk score was only weakly associated with grade in the discovery cohort, and demonstrated higher variation among WHO grade I meningiomas in the validation cohort. Nevertheless, the gene risk score remained significantly prognostic across multiple subgroups.

The study also included both primary and recurrent cases in our discovery and validation cohorts. We chose to do so because such cases are more reflective of the clinical population of meningioma encountered in routine practice, and it is often patients with recurrent disease for whom a prognostic marker would be of utility in guiding adjuvant surveillance or radiotherapy regimens. Further, it is not clear that recurrent or transformed tumors exhibit fundamentally different biology compared to primary meningioma, beyond a greater accumulation of CNVs⁸⁰ and, in general, higher proliferative indices and poorer outcome. Rather than genetic or molecular markers, prior studies have identified a faster time from prior therapy to recurrence and traditional proliferative markers to be most prognostic for recurrent meningioma^{63,66}. Thus, recurrent meningiomas may exist further along the same axis of tumor progression, and their genetic and transcriptional characteristics may in fact be particularly informative as to molecular programs driving clinically aggressive meningiomas. This notion seems to be borne out in our data, as our gene signature remained highly discriminatory within a population of primary and previously untreated meningiomas from our discovery cohort.

### Example 2. Identification of a Second Panel of Genes for Assessing Meningioma

### Methods

A discovery cohort of meningiomas with adequate frozen tissue (N=174) was identified retrospectively from an institutional biorepository and clinical database, as previously described. Our validation cohort for this example was comprised of consecutive meningiomas (N=351) treated at the University of Hong Kong (HKU) between the years 2000 and 2019 with sufficient frozen tissue suitable for molecular analysis. Meningiomas undergoing biopsy only were excluded. Meningiomas were re-reviewed based upon WHO 2016 criteria by an experienced clinical neuro-pathologist. Local failure was defined in cases of gross total resection as appearance of new disease within or immediately adjacent to the resection cavity, and in cases of subtotal resection was defined in the same way or as growth of residual tumor by 25% or more in any dimension on interval MRI. Gross total resection was defined as Simpson Grade I-III resection as determined intraoperatively by the surgeon, or by review of the operative note and post-operative MRI. Primary outcomes of interest were local freedom from recurrence (LFFR), disease specific survival (DSS), and overall survival (OS). The median follow-up was estimated using the reverse Kaplan Meier method. This study was approved by the UCSF Institutional Review Board (IRB #17-22324 and IRB #17-23196).

Details regarding extraction of total RNA and DNA are previously described in detail. For DNA methylation, genomic DNA was processed on the Illumina 850K EPIC beadchip and analyzed using standard procedures to obtain β values W=methylated/[methylated+unmethylated]). K-means consensus clustering was utilized to identify 3 robust DNA methylation groups with distinct molecular and clinical characteristics: Merlin-intact, immune-enriched, and hypermitotic; the stability of these 3 methylation profiles was confirmed by a support vector machine classifier which achieved 97.9% accuracy (95% CI 89.2-99.9%, p<2.2×10⁻¹⁶) in a 25% hold-out test set of meningiomas. RNA sequencing was performed on an Illumina HiSeq 4000 to a mean depth of 42 million reads per sample, and analyzed using standard bioinformatic pipelines, as previously described.

Candidate genes of interest were identified based upon established prognostic significance for meningioma in our previous work or based upon a comprehensive review of the literature, resulting in a rationally designed set of 101 candidate meningioma genes and 25 candidate meningioma-specific housekeeping genes (Table 4). Targeted gene expression profiling was performed of these 125 genes using a custom Nanostring panel. Initial quality control based upon internal negative and spike-in positive controls was performed in the nCounter Analysis System according to the manufacturer's protocol. Next, housekeeping genes were ranked based on noise-to-signal ratio, and 7 optimal housekeeping genes with lowest noise-to-signal encompassing the dynamic range of expression counts were selected. The ratio of geometric means of these 7 housekeeping genes and of the spike-in positive controls was used to assess the adequacy of samples, and samples with a ratio of 0.25 or less (4.5% of samples) were deemed of inadequate quality and excluded from analysis.

Following quality control, a least-absolute shrinkage and selection operator (Lasso) regularized Cox regression model was trained using 10-fold cross validation and the concordance-index (c-index) metric on the resulting discovery dataset (N=173 meningiomas), utilizing the cv.glmnet function in R (Table 5). This analysis resulted in identification of an optimal model containing 34 meningioma genes (FIG. 8, Table 6). As a sanity check, the same approach using an input of 25 housekeeping genes failed to identify any model with prognostic value (data not shown). The resulting continuous risk score was linearly re-scaled between 0 and 1, and an optimal threshold was identified based on the maximally selected rank statistic, resulting in a “low risk” (cutoff=0.461) and a higher risk group. The higher risk group was then subjected a second time to the same procedure to identify a second threshold (cutoff=0.565), resulting in 3 total risk groups, denoted as “low risk”, “intermediate risk”, and “high risk”. All model coefficients and risk score thresholds were then locked and applied without alteration to the validation dataset. The performance of the targeted gene expression risk score was evaluated using standard metrics, including the c-index, Log-rank test, univariate and multivariate Cox regression, and calculation of time-dependent area under the receiver operant curve and Brier error scores. Unless specified, all statistical tests were two-tailed and p values<0.05 were considered significant.

### Results

Targeted gene expression analysis of a discovery dataset of 173 meningiomas (Table 3, FIG. 9) resulted in a 34-gene biomarker (Table 6) and targeted gene expression risk score (FIG. 4A) achieving a c-index of 0.83±0.02 (S.E) for LFFR (FIG. 4B), 0.85±0.04 for DSS (FIGS 13), and 0.77±0.04 for OS (FIG. 4B). Application of this biomarker to an independently collected external validation cohort of 331 meningiomas (Table 3, FIG. 10) resulted in a well-distributed targeted gene expression risk score (FIG. 4C) achieving a c-index of 0.75±0.03 for LFFR (FIG. 4C), 0.79±0.04 for DSS (FIG. 13), and 0.72±0.03 for OS (FIG. 4C), significantly outperforming WHO grade (c-index for LFFR: 0.65±0.03, bootstrap delta-AUC for LFFR at 5 years: 0.11, 95% CI 0.063-0.156). The prognostic performance of the gene expression risk score was comprehensively investigated across clinical contexts, common copy-number alteration (CNA) subgroups (Ch1p and Ch22q), and previously identified DNA methylation groups, corroborating the independent prognostic value of the biomarker across both clinical and molecular strata (FIG. 5, FIG. 11, FIG. 12). Multivariable Cox regression adjusting for clinical covariates (WHO grade, extent of resection, setting, adjuvant radiation), CNA status, and methylation group confirmed the independent prognostic value of the biomarker for LFFR, DSS, and OS (FIG. 5, FIG. 15).

Next, the predictive value of the targeted gene expression risk score was evaluated in the context of adjuvant radiotherapy. Among WHO grade 2 tumors, a clinical subgroup for whom adjuvant radiotherapy remains controversial and which is the subject of two ongoing randomized trials, the targeted gene expression risk score was predictive in identifying a subset of tumors that benefited from adjuvant radiotherapy (FIG. 6A), with a HR of 0.54 (95% CI 0.3-1.0, p=0.0495) among intermediate and high risk tumors versus 1.0 (95% CI 0.2-7.2, p=0.97) among low risk tumors. Among a propensity matched cohort (N=76, matched on WHO grade, EOR, setting, MIB labeling index, methylation group, Ch1p status, and Ch22q status) of meningiomas with or without adjuvant radiotherapy, the targeted gene expression risk score was similarly predictive (FIG. 6B). In contrast, neither WHO grade nor methylation group was predictive for radiotherapy response (FIG. 14). Based upon risk factors used for selection of patients for radiotherapy in the phase II trial RTOG 0539, use of the targeted gene expression risk score would have resulted in a potential change of management in 30.2% of patients (N=100 of 331, FIG. 4C), including 35% (N=19 of 55) of patients within RTOG 0539's “intermediate” risk strata who may have avoided adjuvant radiotherapy based on our biomarker.

Finally, in order to facilitate the clinical application of our targeted gene expression biomarker, multivariable Cox models were created incorporating practical clinical covariates (WHO grade, EOR, setting, and adjuvant radiotherapy) with the addition of the gene expression biomarker, which resulted in a well calibrated model (FIG. 16) achieving an AUC for 5 year LFFR of 0.81 within the validation dataset (FIG. 6A-D), comparing favorably to a similar model using DNA methylation groups and outperforming the standard of care using WHO grade (AUC 0.75). A clinical nomogram is displayed demonstrating the potential utility of our continuous targeted gene expression risk score in improving the risk stratification and personalized management of meningiomas.

### Discussion of Results Obtained in Example 2

Here, we use a targeted gene expression approach to identify and externally validate a clinically tractable 34-gene biomarker for meningioma risk stratification, demonstrating its independent prognostic value across clinical and molecular contexts, and establishing its potential role in personalizing the post-surgical management of patients with meningioma.

Strengths of the present biomarker and report include the favorable cost, logistic simplicity, and well-established characteristics of a continuous targeted gene expression risk score, an approach which has been applied and repeatedly validated with success in other clinical contexts, particularly in breast and prostate cancer. Further, the present study reports one of the largest independent meningioma validation cohorts from an external, international center providing the majority of neurosurgical care for a large local population, resulting in a well-distributed cohort of meningioma patients more representative of a “typical” population, thus reducing the potential for selection bias. Our biomarker panel has robust discriminative power across multiple contexts, importantly demonstrating independent prognostic value within methylation and copy number alteration strata, and after adjusting for these molecular characteristics as well as established clinical covariates. Whereas prior reports of prognostic DNA methylation and transcriptome-based profiling reported smaller validation cohorts in which WHO grade and clinical covariates achieved lower discriminative power than would be expected in routine clinical care, possibly owing to selection bias among meningiomas treated at tertiary academic centers, our biomarker demonstrated substantial additive prognostic value when combined with WHO grade and clinical covariates in a well-distributed validation cohort in which WHO grade and clinical variables were already reasonably prognostic. These performance characteristics and the rate of tumor risk reclassification of 30.2% compare favorably to similar, well-established biomarkers already in routine clinical use for breast and prostate cancer patients.

The inexpensive and clinically tractable targeted meningioma gene expression panel signature identified was independently prognostic for local failure, disease specific mortality, and overall survival after surgery across clinical, DNA methylation, and copy number alteration contexts, and was predictive for benefit from adjuvant radiotherapy in an independent, external, retrospective cohort. Prospective trials incorporating this biomarker for risk stratification are warranted.

### Summary of Example 1 and 2

Gene signature panels and prognostic risk scores identified in Examples 1 and 2 based on targeted gene expression analysis of meningiomas significantly outperformed WHO grade in stratifying cases by local freedom from recurrence and overall survival, and may be useful for guiding surveillance or adjuvant therapy after surgery.

### LISTING OF REFERENCES CITED BY NUMBER

1. Ostrom Q T, Gittleman H, Truitt G, Boscia A, Kruchko C, Barnholtz-Sloan J S. CBTRUS statistical report: Primary brain and other central nervous system tumors diagnosed in the United States in 2011-2015. Neuro Oncol. 2018. doi:10.1093/neuonc/noy131

2. Aizer A A, Bi W L, Kandola M S, et al. Extent of resection and overall survival for patients with atypical and malignant meningioma. Cancer. 2015. doi:10.1002/cncr.29639

3. Chen W C, Magill S T, Wu A, et al. Histopathologic features predictive of local control of atypical meningioma after surgery and adjuvant radiotherapy. J Neurosurg. 2018; Epub ahead(doi: 10.3171/2017.9.JNS171609.).

4. Condra K S, Buatti J M, Mendenhall W M, Friedman W A, Marcus R B, Rhoton A L. Benign meningiomas: Primary treatment selection affects survival. Int J Radiat Oncol Biol Phys. 1997. doi:10.1016/S0360-3016(97)00317-9

5. Cahill K S, Claus E B. Treatment and survival of patients with nonmalignant intracranial meningioma: Results from the Surveillance, Epidemiology, and End Results Program of the National Cancer Institute—Clinical article. J Neurosurg. 2011. doi:10.3171/2011.3JNS101748

6. Louis D N, Perry A, Reifenberger G, et al. The 2016 World Health Organization Classification of Tumors of the Central Nervous System: a summary. Acta Neuropathol. 2016; 131(6):803-820. doi:10.1007/s00401-016-1545-1

7. Olar A, Wani K M, Wilson C D, et al. Global epigenetic profiling identifies methylation subgroups associated with recurrence-free survival in meningioma. Acta Neuropathol. 2017. doi:10.1007/s00401-017-1678-x

8. Sahm F, Schrimpf D, Stichel D, et al. DNA methylation-based classification and grading system for meningioma: a multicentre, retrospective analysis. Lancet Oncol. 2017; 18(5):682-694. doi:10.1016/S1470-2045(17)30155-9

9. Nassiri F, Mamatjan Y, Suppiah S, et al. DNA methylation profiling to predict recurrence risk in meningioma: Development and validation of a nomogram to optimize clinical management. Neuro Oncol. 2019. doi:10.1093/neuonc/noz061

10. Patel A J, Wan Y W, Al-Ouran R, et al. Molecular profiling predicts meningioma recurrence and reveals loss of DREAM complex repression in aggressive tumors. Proc Natl Acad Sci USA. 2019. doi:10.1073/pnas.1912858116

11. Viaene A N, Zhang B, Martinez-Lage M, et al. Transcriptome signatures associated with meningioma progression. Acta Neuropathol Commun. 2019. doi:10.1186/s40478-019-0690-x

12. Rogers L, Zhang P, Vogelbaum M a., et al. RTOG 0539A: Intermediate-Risk Meningioma: Initial Outcomes from NRG Oncology/RTOG-0539. Astro. 2015; 93(3):S139-S140. doi:10.1016/j.ijrobp.2015.07.331

13. Weber D C, Ares C, Villa S, et al. Adjuvant postoperative high-dose radiotherapy for atypical and malignant meningioma: A phase-II parallel non-randomized and observation study (EORTC 22042-26042). Radiother Oncol. 2018. doi:10.1016/j.radonc.2018.06.018

14. NCT03180268. Observation or Radiation Therapy in Treating Patients With Newly Diagnosed Grade II Meningioma That Has Been Completely Removed by Surgery. https://clinicaltrials.gov/show/NCT03180268.2017.

15. Jenkinson M D, Javadpour M, Haylock B J, et al. The ROAM/EORTC-1308 trial: Radiation versus Observation following surgical resection of Atypical Meningioma: Study protocol for a randomised controlled trial. Trials. 2015. doi:10.1186/s13063-015-1040-3

16. Sahm F, Schrimpf D, Stichel D, et al. DNA methylation-based classification and grading system for meningioma: a multicentre, retrospective analysis. Lancet Oncol. 2017; 2045(17). doi:10.1016/S1470-2045(17)30155-9

17. Preusser M, Brastianos P K, Mawrin C. Advances in meningioma genetics: Novel therapeutic opportunities. Nat Rev Neurol. 2018. doi:10.1038/nrneurol.2017.168

18. Clark V E, Erson-Omay E Z, Serin A, et al. Genomic analysis of non-NF2 meningiomas reveals mutations in TRAF7, KLF4, AKT1, and SMO. Science. 2013; 339(6123):1077-1080. doi:10.1126/science.1233009

19. Clark V E, Harmancl A S, Bai H, et al. Recurrent somatic mutations in POLR2A define a distinct subset of meningiomas. Nat Genet. 2016. doi:10.1038/ng.3651

20. Sahm F, Schrimpf D, Olar A, et al. TERT Promoter Mutations and Risk of Recurrence in Meningioma. J Natl Cancer Inst. 2016. doi:10.1093/jnci/djv377

21. Shankar G M, Santagata S. BAP1 mutations in high-grade meningioma: Implications for patient care. Neuro Oncol. 2017. doi:10.1093/neuonc/nox094

22. Bi W L, Abedalthagafi M, Horowitz P, et al. Genomic landscape of intracranial meningiomas. J Neurosurg. 2016. doi:10.3171/2015.6.JNS15591

23. Vasudevan H N, Braunstein S E, Phillips J J, et al. Comprehensive Molecular Profiling Identifies FOXM1 as a Key Transcription Factor for Meningioma Proliferation. Cell Rep. 2018. doi:10.1016/j.celrep.2018.03.013

24. Horak P, Fröhling S, Glimm H. Integrating next-generation sequencing into clinical oncology: Strategies, promises and pitfalls. ESMO Open. 2016. doi:10.1136/esmoopen-2016-000094

25. Locke W J, Guanzon D, Ma C, et al. DNA Methylation Cancer Biomarkers: Translation to the Clinic. Front Genet. 2019. doi:10.3389/fgene.2019.01150

26. Sun S Q, Cai C, Murphy R K J, et al. Radiation therapy for residual or recurrent atypical meningioma: The effects of modality, timing, and tumor pathology on long-term outcomes. Neurosurgery. 2016; 79(1):23-32. doi:10.1227/NEU.0000000000001160

27. Olar A, Wani K M, Sulman E P, et al. Mitotic index is an independent predictor of recurrence-free survival in meningioma. In: Brain Pathology. Vol 25; 2015:266-275. doi:10.1111/bpa.12174

28. Bruna J, Brell M, Ferrer I, Gimenez-Bonafe P, Tortosa A. Ki-67 proliferative index predicts clinical outcome in patients with atypical or anaplastic meningioma. Neuropathology. 2007; 27(2):114-120. doi:10.1111/j.1440-1789.2007.00750.x

29. Sparano J A, Gray R J, Makower D F, et al. Adjuvant chemotherapy guided by a 21-gene expression assay in breast cancer. N Engl J Med. 2018. doi:10.1056/NEJMoa1804710

30. Knezevic D, Goddard A D, Natraj N, et al. Analytical validation of the Oncotype DX prostate cancer assay—a clinical RT-PCR assay optimized for prostate needle biopsies. BMC Genomics. 2013. doi:10.1186/1471-2164-14-690

31. Cuzick J, Berney D M, Fisher G, et al. Prognostic value of a cell cycle progression signature for prostate cancer death in a conservatively managed needle biopsy cohort. Br J Cancer. 2012. doi:10.1038/bjc.2012.39

32. Lee Y, Liu J, Patel S, et al. Genomic landscape of meningiomas. Brain Pathol. 2010. doi:10.1111/j.1750-3639.2009.00356.x 33. Tibshirani R, Hastie T, Narasimhan B, Chu G. Diagnosis of multiple cancer types by shrunken centroids of gene expression. Proc Natl Acad Sci USA. 2002. doi:10.1073/pnas.082099299

34. Hastie T, Tibshirani R, Narasimhan B, Chu G. pamr: Pam: Prediction Analysis for Microarrays. 2019. https://cran.r-project.org/package=pamr.

35. Hofree M, Shen J P, Carter H, Gross A, Ideker T. Network-based stratification of tumor mutations. Nat Methods. 2013. doi:10.1038/nmeth.2651

36. Alexander T B, Gu Z, Iacobucci I, et al. The genetic basis and cell of origin of mixed phenotype acute leukaemia. Nature. 2018. doi:10.1038/s41586-018-0436-0

37. De Sousa E Melo F, Wang X, Jansen M, et al. Poor-prognosis colon cancer is defined by a molecularly distinct subtype and develops from serrated precursor lesions. Nat Med. 2013. doi:10.1038/nm.3174

38. Kishan K, Rui L, Feng C, Qi Y, Haake A R. GNE: A deep learning framework for gene network inference by aggregating biological information. BMC Syst Biol. 2019. doi:10.1186/512918-019-0694-y

39. Pedrogosa F, Varoquaux G, Gramfort A, Al E. Scikit-learn: Machine Learning in Python. J Mach Learn Res. 2011; 12((October)):2825-2830.

40. Lee Y, Liu J, Patel S, et al. Genomic landscape of meningiomas. Brain Pathol. 2010; 20(4):751-762. doi:10.1111/j.1750-3639.2009.00356.x

41. Gentleman R C, Carey V J, Bates D M, et al. Bioconductor: open software development for computational biology and bioinformatics. Genome Biol. 2004. doi:10.1186/gb-2004-5-10-r80

42. Kamburov A, Wierling C, Lehrach H, Herwig R. ConsensusPathDB—A database for integrating human functional interaction networks. Nucleic Acids Res. 2009. doi:10.1093/nar/gkn698

43. Szklarczyk D, Gable A L, Lyon D, et al. STRING v11: Protein-protein association networks with increased coverage, supporting functional discovery in genome-wide experimental datasets. Nucleic Acids Res. 2019. doi:10.1093/nar/gky1131

44. Liao G Bin, Li X Z, Zeng S, et al. Regulation of the master regulator FOXM1 in cancer. Cell Commun Signal. 2018. doi:10.1186/s12964-018-0266-6

45. Obexer P, Hagenbuchner J, Unterkircher T, et al. Repression of BIRC5/Survivin by FOXO3/FKHRL1 sensitizes human neuroblastoma cells to DNA damage-induced apoptosis. Mol Biol Cell. 2009. doi:10.1091/mbc.E08-07-0699

46. Nitiss J L. Targeting DNA topoisomerase II in cancer chemotherapy. Nat Rev Cancer. 2009. doi:10.1038/nrc2607

47. Yuan J, Yan R, Krämer A, et al. Cyclin B1 depletion inhibits proliferation and induces apoptosis in human tumor cells. Oncogene. 2004. doi:10.1038/sj.onc.1207757

48. Liang C J, Wang Z W, Chang Y W, Lee K C, Lin W H, Lee J L. SFRPs Are Biphasic Modulators of Wnt-Signaling-Elicited Cancer Stem Cell Properties beyond Extracellular Control. Cell Rep. 2019. doi:10.1016/j.celrep.2019.07.023

49. Musa J, Aynaud M M, Mirabeau O, Delattre O, Grünewald T G. MYBL2 (B-Myb): a central regulator of cell proliferation, cell survival and differentiation involved in tumorigenesis. Cell Death Dis. 2017. doi:10.1038/cddis.2017.244

50. Kallioniemi A. Bone morphogenetic protein 4-a fascinating regulator of cancer cell behavior. Cancer Genet. 2012. doi:10.1016/j.cancergen.2012.05.009

51. Braig S, Wallner S, Junglas B, Fuchshofer R, Bosserhoff A K. CTGF is overexpressed in malignant melanoma and promotes cell invasion and migration. Br J Cancer. 2011. doi:10.1038/bjc.2011.226

52. Del Sal G, et al. The growth arrest-specific gene, gas1, is involved in growth suppression. Cell. 1992. doi:10.1016/0092-8674(92)90429-G

53. Wolfsberger S, Doostkam S, Boecher-Schwarz H G, et al. Progesterone-receptor index in meningiomas: Correlation with clinico-pathological parameters and review of the literature. Neurosurg Rev. 2004. doi:10.1007/s10143-004-0340-y

54. Pérez-Magán E, De Lope Á R, Ribalta T, et al. Differential expression profiling analyses identifies downregulation of 1p, 6q, and 14q genes and overexpression of 6p histone cluster 1 genes as markers of recurrence in meningiomas. Neuro Oncol. 2010. doi:10.1093/neuonc/noq081

55. Pérez-Magán E, Campos-Martín Y, Mur P, et al. Genetic alterations associated with progression and recurrence in meningiomas. J Neuropathol Exp Neurol. 2012. doi:10.1097/NEN.0b013 e31826bf704

56. Kotecha R S, Pascoe E M, Rushing E J, et al. Meningiomas in children and adolescents: A meta-analysis of individual patient data. Lancet Oncol. 2011. doi:10.1016/S1470-2045(11)70275-3

57. Kotecha R S, Jacoby P, Cole C H, Gottardo N G. Morbidity in survivors of child and adolescent meningioma. Cancer. 2013. doi:10.1002/cncr.28366

58. Van Alkemade H, De Leau M, Dieleman E M T, et al. Impaired survival and long-term neurological problems in benign meningioma. Neuro Oncol. 2012. doi:10.1093/neuonc/nos013

59. Chen W C, Magill S T, Wu A, et al. Histopathological features predictive of local control of atypical meningioma after surgery and adjuvant radiotherapy. J N eurosurg. 2018. doi:10.3171/2017.9.jns171609

60. Chen W C, Magill S T, Wu A, et al. Histopathological features predictive of local control of atypical meningioma after surgery and adjuvant radiotherapy. J Neurosurg. 2019. doi:10.3171/2017.9.JNS171609

61. Aizer A A, Arvold N D, Catalano P, et al. Adjuvant radiation therapy, local recurrence, and the need for salvage therapy in atypical meningioma. Neuro Oncol. 2014; 16(11):1547-1553. doi:10.1093/neuonc/nou098

62. Adeberg S, Hartmann C, Welzel T, et al. Long-Term Outcome After Radiotherapy in Patients With Atypical and Malignant Meningiomas—Clinical Results in 85 Patients Treated in a Single Institution Leading to Optimized Guidelines for Early Radiation Therapy. Int J Radiat Oncol. 2012; 83(3):859-864. doi:10.1016/j.ijrobp.2011.08.010

63. Bagshaw H P, Burt L M, Jensen R L, et al. Adjuvant radiotherapy for atypical meningiomas. J Neurosurg. 2016:1-7. doi:10.3171/2016.5.JNS152809

64. Hammouche S, Clark S, Wong A H L, Eldridge P, Farah J O. Long-term survival analysis of atypical meningiomas: Survival rates, prognostic factors, operative and radiotherapy treatment. Acta Neurochir (Wien). 2014; 156(8):1475-1481. doi:10.1007/s00701-014-2156-z

65. Komotar R J, Iorgulescu J B, Raper D M S, et al. The role of radiotherapy following gross-total resection of atypical meningiomas. J Neurosurg. 2012; 117(4):679-686. doi:10.3171/2012.7.JNS112113

66. Chen W C, Hara J, Magill S T, et al. Salvage therapy outcomes for atypical meningioma. J Neurooncol. 2018. doi:10.1007/s11060-018-2813-9

67. Magill S T, Lee D S, Yen A J, et al. Surgical outcomes after reoperation for recurrent skull base meningiomas. J Neurosurg. 2019. doi:10.3171/2017.11.JNS172278

68. Kaur G, Sayegh E T, Larson A, et al. Adjuvant radiotherapy for atypical and malignant meningiomas: A systematic review. Neuro Oncol. 2014; 16(5):628-636. doi:10.1093/neuonc/nou025

69. Najafabadi A, Van Der Meer P, Boele F, et al. The long-term disease burden of meningioma patients: results on health-related quality of life, cognitive function, anxiety and depression. Neuro Oncol. 2018; 20(suppl_6):vi154-vi155. doi:https://doi.org/10.1093/neuonc/noy148.643

70. Pollock B E, Link M J, Stafford S L, Parney I F, Garces Y I, Foote R L. The Risk of Radiation-Induced Tumors or Malignant Transformation After Single-Fraction Intracranial Radiosurgery: Results Based on a 25-Year Experience. Int J Radiat Oncol Biol Phys. 2017. doi:10.1016/j.ijrobp.2017.01.004

71. Laurendeau I, Ferrer M, Garrido D, et al. Gene expression profiling of the hedgehog signaling pathway in human meningiomas. Mol Med. 2010. doi:10.2119/molmed.2010.00005

72. Kim H, Park K J, Ryu B K, et al. Forkhead box M1 (FOXM1) transcription factor is a key oncogenic driver of aggressive human meningioma progression. Neuropathol Appl Neurobiol. 2019. doi:10.1111/nan.12571

73. S. H. K, H. K, K. P, B. R, M. Y, Y. C. Altered FOXM1 expression contributes to the meningioma malignancy and can be a critical target for the tumor progression. Neuro Oncol. 2018. doi:http://dx.doi.org/10.1093/neuonc/noy139

74. Stuart J E, Lusis E A, Scheck A C, et al. Identification of gene markers associated with aggressive meningioma by filtering across multiple sets of gene expression arrays. J Neuropathol Exp Neurol. 2011. doi:10.1097/NEN.0b013e3182018f1c

75. de Moraes G N, Delbue D, Silva K L, et al. FOXM1 targets XIAP and Survivin to modulate breast cancer survival and chemoresistance. Cell Signal. 2015. doi:10.1016/j.cellsig.2015.09.013

76. Roser F, Nakamura M, Bellinzona M, Rosahl S K, Ostertag H, Samii M. The prognostic value of progesterone receptor status in meningiomas. J Clin Pathol. 2004. doi:10.1136/jcp.2004.018333

77. Lusis E A, Watson M A, Chicoine M R, et al. Integrative genomic analysis identifies NDRG2 as a candidate tumor suppressor gene frequently inactivated in clinically aggressive meningioma. Cancer Res. 2005. doi:10.1158/0008-5472.CAN-05-0043

78. Rajaraman P, Brenner A V., Neta G, et al. Risk of meningioma and common variation in genes related to innate immunity. Cancer Epidemiol Biomarkers Prey. 2010. doi:10.1158/1055-9965.EPI-09-1151

79. Carvalho L H, Smirnov I, Baia G S, et al. Molecular signatures define two main classes of meningiomas. Mol Cancer. 2007. doi:10.1186/1476-4598-6-64

80. Krayenbühl N, Pravdenkova S, Al-Mefty O. De novo versus transformed atypical and anaplastic meningiomas: Comparisons of clinical course, cytogenetics, cytokinetics, and outcome. Neurosurgery. 2007. doi:10.1227/01.NEU.0000290895.92695.22

It is understood that the examples and embodiments described herein are for illustrative purposes only and that various modifications or changes in light thereof will be suggested to persons skilled in the art and are to be included within the spirit and purview of this application and scope of the appended claims. All publications, patents, accession numbers, and patent applications cited herein are hereby incorporated by reference in their entirety for all purposes.

TABLE 1
Meningioma gene signature discovery and validation
cohort characteristics for Example 1.

| | |
|---|---|
| Discovery cohort | Validation cohort |

Clinical Characteristic by Patient

| Number of unique patients | 84 | | 56 | |
|---|---|---|---|---|
| Median patient age | 60.4y | (IQR 52.6-67.6) | 64.0 | (53.0-76.0) |
| Number of male patients | 33 | (39%) | 23 | (41%) |
| Number of patients with recurrent | 22 | (26%) | 11 | (20%) |
| meningioma at presentation | | | | |
| Number of patients with prior | 15 | (18%) | Unknown | |
| meningioma resections | | | | |
| Number of patients with prior | 18 | (21%) | 5 | (9%) |
| meningioma radiotherapy | | | | |
| Number of gross total resections | 39 | (46%) | 47 | (84%) |
| WHO grade of first resection | | | | |
| I | 12 | (14%) | 35 | (63%) |
| II | 64 | (76%) | 16 | (29%) |
| III | 7 | (8%) | 5 | (9%) |
| Median clinical follow up (years) | 6.4 | (IQR 3.6-9.1) | 5.4 | (2.9-7.0) |
| Number of patients who died | 35 | (42%) | 10 | (18%) |

| | | | |
|---|---|---|---|
| Number of patients who died | 17 | (20%) | Unknown |
| of meningioma progression | | | |

Clinical Characteristics by Meningioma

| Number of meningiomas | 96 | | 56 | |
|---|---|---|---|---|
| Number of meningiomas | 33 | (34%) | 11 | (20%) |
| recurrent at presentation | | | | |
| Number of meningiomas with prior surgery | 19 | (20%) | Unknown | |
| Number of meningiomas with prior | 29 | (30%) | 5 | (9%) |
| radiotherapy | | | | |
| Number of gross total resections | 45 | (47%) | 47 | (84%) |
| WHO grade | | | | |
| I | 13 | (14%) | 35 | (63%) |
| II | 64 | (67%) | 16 | (29%) |
| III | 19 | (20%) | 5 | (9%) |
| Number recurred | 49 | (58%) | 11 | (20%) |

TABLE 2
“CHRLOC” and “CHRLOCEND” refer to start and end positions
of each gene. Genes were mapped to “Genome Reference Consortium
Human Build 38”, GRCh38, which the code accessed on Mar. 13, 2018. (Example 1)

| | | | | | Univariate | |
|---|---|---|---|---|---|---|
| | | | | | Cox, discovery | Univariate |
| | | | | Univariate | cohort, LFFR, | Cox, discovery |
| | | | | Cox, discovery | Bonferroni | cohort, LFFR, |
| | | | | cohort, LFFR, | corrected | Beta- |
| SYMBOL | CHR | CHRLOC | CHRLOCEND | p-value | p-value | coefficient |
| CD34 | 1 | −207886537 | −207911338 | 0.20071542 | 54.5945938 | −0.5506023 |
| PTGS2 | 1 | −186671811 | −186680427 | 0.67275143 | 182.988389 | 0.17313065 |
| TPR | 1 | −186311653 | −186375325 | 0.03421651 | 9.30689133 | 1.65710938 |
| MUC1 | 1 | −155185823 | −155192915 | 0.76361672 | 207.703749 | 0.1399476 |
| S100A4 | 1 | −153543621 | −153545806 | 0.17024662 | 46.3070802 | 0.55067805 |
| MCL1 | 1 | −150574550 | −150579738 | 0.52535874 | 142.897578 | −0.4875303 |
| NRAS | 1 | −114704463 | −114716894 | 0.01839783 | 5.00421003 | 1.85251565 |
| BCAR3 | 1 | −93561785 | −93681370 | 0.04535536 | 12.3366588 | −0.807779 |
| TGFBR3 | 1 | −91680342 | −91886279 | 0.00066655 | 0.18130146 | −1.6351427 |
| FUBP1 | 1 | −77948401 | −77979205 | 0.55824105 | 151.841567 | −0.5694 |
| JUN | 1 | −58780790 | −58784113 | 0.70636566 | 192.131459 | −0.1381731 |
| TAL1 | 1 | −47216289 | −47232335 | 0.01815992 | 4.93949761 | −0.9230412 |
| MYCL1 | 1 | −39895426 | −39902256 | 0.00531703 | 1.446231 | −1.2880524 |
| CSF3R | 1 | −36466042 | −36483314 | 0.00850148 | 2.31240364 | −1.3275136 |
| SFPQ | 1 | −35183599 | −35193142 | 0.60279396 | 163.959957 | −0.5700273 |
| FGR | 1 | −27612289 | −27626595 | 0.00341973 | 0.93016764 | −1.3153294 |
| PLA2G2A | 1 | −19975430 | −19980439 | 0.4843585 | 131.745513 | 0.13899333 |
| TNFRSF1B | 1 | 12167002 | 12209220 | 0.00904353 | 2.45984079 | −1.3311268 |
| LCK | 1 | 32251238 | 32286167 | 0.60738283 | 165.208129 | −0.2358561 |
| HDAC1 | 1 | 32292106 | 32333623 | 0.00107573 | 0.2925983 | −2.2638854 |
| MPL | 1 | 43337803 | 43354464 | 0.00618629 | 1.68267167 | −1.5846507 |
| RAD54L | 1 | 46247694 | 46278473 | 0.00449876 | 1.22366172 | 1.99599063 |
| CDKN2C | 1 | 50968694 | 50974634 | 0.79925689 | 217.397873 | −0.1028016 |
| PPAP2B | 1 | 56494761 | 56645301 | 0.02885751 | 7.84924223 | −0.8392517 |
| GADD45A | 1 | 67685176 | 67688338 | 0.407433 | 110.821776 | −0.4008504 |
| CYR61 | 1 | 85580760 | 85583965 | 0.00448298 | 1.21937133 | −0.785565 |
| LMO4 | 1 | 87328467 | 87348923 | 0.52823594 | 143.680175 | −0.197498 |
| DAP3 | 1 | 155689090 | 155739009 | 0.06327628 | 17.2111489 | 1.17874144 |
| NTRK1 | 1 | 156815749 | 156881850 | 0.18837469 | 51.2379162 | 0.74929074 |
| ERBB4 | 2 | −211375716 | −212538628 | 0.99934051 | 271.820617 | 0.00027956 |
| STAT1 | 2 | −190975536 | −191014250 | 0.09241035 | 25.1356153 | 0.80242007 |
| FRZB | 2 | −182833275 | −182866770 | 0.02057631 | 5.59675755 | −0.5350871 |
| IL1B | 2 | −112829759 | −112836779 | 0.12523267 | 34.0632854 | 0.47913924 |
| IL1A | 2 | −112773914 | −112785398 | 0.00641793 | 1.74567804 | 1.12427488 |
| TGFA | 2 | −70447279 | −70553638 | 0.03605299 | 9.80641312 | −1.5459254 |
| MYCN | 2 | 15940437 | 15947007 | 0.22446356 | 61.0540895 | 0.62662235 |
| FOSL2 | 2 | 28392911 | 28414649 | 0.246209 | 66.9688475 | −0.4224424 |
| MSH2 | 2 | 47403066 | 47483228 | 0.12808533 | 34.8392105 | 1.16385232 |
| MSH6 | 2 | 47783081 | 47806953 | 0.14980128 | 40.745949 | 1.4387636 |
| REL | 2 | 60881494 | 60928156 | 0.00254581 | 0.69246026 | −2.2717413 |
| CASP10 | 2 | 201182880 | 201200729 | 0.01185789 | 3.22534693 | −1.9966337 |
| XRCC5 | 2 | 216109296 | 216206293 | 0.36314231 | 98.7747092 | 1.09166911 |
| IGFBP2 | 2 | 216632827 | 216664435 | 0.57458123 | 156.286093 | 0.20227574 |
| TFRC | 3 | −196049283 | −196082090 | 0.00159933 | 0.43501804 | 1.28769315 |
| BCL6 | 3 | −187721376 | −187736497 | 0.04360502 | 11.8605668 | −1.5394727 |
| THPO | 3 | −184371934 | −184378208 | 0.47646759 | 129.599186 | −0.4859558 |
| TNFSF10 | 3 | −172517354 | −172523475 | 0.18701645 | 50.8684747 | −0.4103289 |
| MST1R | 3 | −49887002 | −49903637 | 0.2159082 | 58.7270317 | 0.58696944 |
| XPC | 3 | −14145147 | −14178672 | 0.35815556 | 97.4183136 | −0.9987997 |
| RAF1 | 3 | −12583600 | −12664201 | 0.18731829 | 50.9505759 | 1.64313259 |
| OGG1 | 3 | 9749943 | 9766669 | 0.12176212 | 33.1192972 | 1.39919136 |
| PPARG | 3 | 12287484 | 12434356 | 0.08078421 | 21.9733045 | −1.035113 |
| TGFBR2 | 3 | 30606501 | 30694141 | 0.06491473 | 17.6568068 | −1.1800697 |
| MLH1 | 3 | 36993349 | 37050846 | 0.41093691 | 111.774839 | −0.9060416 |
| CTNNB1 | 3 | 41199423 | 41240453 | 0.82969463 | 225.676939 | 0.16025405 |
| PTPRG | 3 | 61561568 | 62294898 | 0.39220545 | 106.679882 | −0.5022657 |
| ZIC1 | 3 | 147409393 | 147416719 | 0.09343141 | 25.4133442 | −0.4205146 |
| PIK3CA | 3 | 179148522 | 179240093 | 0.025645 | 6.97544051 | −1.8746687 |
| FAT1 | 4 | −186587782 | −186723833 | 0.04507502 | 12.2604053 | 1.37754115 |
| CCNA2 | 4 | −121816443 | −121824001 | 0.00055686 | 0.15146515 | 1.77140209 |
| MAPK10 | 4 | −86012295 | −86360222 | 0.2815054 | 76.5694678 | −0.4371355 |
| CXCL9 | 4 | −76001341 | −76007523 | 0.19425465 | 52.837265 | 0.33271574 |
| KDR | 4 | −55078258 | −55125595 | 0.00314387 | 0.85513226 | −1.1765554 |
| FGFR3 | 4 | 1793311 | 1808872 | 0.26361855 | 71.7042453 | −0.3141874 |
| UCHL1 | 4 | 41256880 | 41268429 | 0.91139557 | 247.899596 | −0.0222625 |
| PDGFRA | 4 | 54229088 | 54298247 | 0.34640338 | 94.22172 | 0.21475017 |
| KIT | 4 | 54657927 | 54740715 | 0.0580046 | 15.7772502 | −0.7428479 |
| IL8 | 4 | 73740541 | 73743716 | 0.00202838 | 0.55171845 | 0.72214933 |
| AREG | 4 | 74445097 | 74455009 | 0.19955058 | 54.2777572 | −0.5052246 |
| SPP1 | 4 | 87975649 | 87983411 | 0.00624613 | 1.69894711 | 0.56440103 |
| EGF | 4 | 109912883 | 110013079 | 0.0328124 | 8.92497332 | 1.57917414 |
| FGF2 | 4 | 122826707 | 122898235 | 0.0141985 | 3.86199141 | −0.7373547 |
| PDGFRB | 5 | −150152338 | −150155859 | 0.96919619 | 263.621363 | −0.0184443 |
| CSF1R | 5 | −150053290 | −150086703 | 0.04058134 | 11.0381238 | −0.9084875 |
| FGF1 | 5 | −142592177 | −142686495 | 0.61754643 | 167.972628 | −0.1927047 |
| CDC25C | 5 | −138285264 | −138331877 | 0.00022504 | 0.06121098 | 1.68915755 |
| IRF1 | 5 | −132489112 | −132490773 | 0.34337976 | 93.3992958 | 0.47381274 |
| TERT | 5 | −1253171 | −1295047 | 0.17617455 | 47.9194772 | 0.6582018 |
| APC | 5 | 112707504 | 112846239 | 0.19190701 | 52.198708 | 1.15322169 |
| IL4 | 5 | 132673985 | 132682678 | 0.24004923 | 65.2933898 | −0.60938 |
| TGFBI | 5 | 136028894 | 136063818 | 0.66780326 | 181.642486 | −0.1235181 |
| EGR1 | 5 | 138465491 | 138469315 | 0.63624122 | 173.057613 | 0.11026109 |
| HMMR | 5 | 163460510 | 163491946 | 0.00445803 | 1.21258457 | 1.77683424 |
| NPM1 | 5 | 171387115 | 171410884 | 0.531382 | 144.535904 | 0.34104955 |
| FGFR4 | 5 | 177086871 | 177098142 | 0.53643904 | 145.911418 | 0.29937997 |
| DLL1 | 6 | −170282205 | −170290609 | 0.00031191 | 0.08483866 | −1.5057465 |
| IFNGR1 | 6 | −137197483 | −137219430 | 0.3189007 | 86.7409914 | −0.5608611 |
| CTGF | 6 | −131948176 | −131951378 | 0.01938815 | 5.27357754 | −0.5982604 |
| FYN | 6 | −111660331 | −111873452 | 0.08611556 | 23.4234323 | −0.6380487 |
| CCND3 | 6 | −41934932 | −41941848 | 0.05249395 | 14.2783544 | −1.1098947 |
| DEK | 6 | −18224168 | −18264568 | 0.92947502 | 252.817204 | −0.0601324 |
| FOXC1 | 6 | 1610445 | 1613894 | 0.78848528 | 214.467997 | 0.14909825 |
| TUBB | 6 | 1975312 | 1980539 | 0.04144938 | 11.2742308 | 1.65965037 |
| TNF | 6 | 2823299 | 2826068 | 0.08640882 | 23.5031989 | 0.86647818 |
| E2F3 | 6 | 20401905 | 20493714 | 0.32404712 | 88.140817 | 0.71529284 |
| CDKN1A | 6 | 36676459 | 36687339 | 0.44715032 | 121.624887 | −0.2994646 |
| PIM1 | 6 | 37170145 | 37175428 | 0.00079006 | 0.21489714 | 1.29640378 |
| PTK7 | 6 | 43076267 | 43161720 | 0.70940094 | 192.957056 | 0.18454374 |
| HSP90AB1 | 6 | 44246165 | 44253888 | 0.21340562 | 58.0463275 | −0.9733075 |
| FOXO3A | 6 | 108559835 | 108,684,774 | 0.00487631 | 1.32635613 | −1.4857604 |
| MYB | 6 | 135181314 | 135219173 | 0.60113972 | 163.510002 | −0.3597659 |
| ESR1 | 6 | 151690495 | 152103273 | 0.19609007 | 53.3364993 | −0.7688346 |
| PLG | 6 | 160702192 | 160754053 | 0.44450188 | 120.904512 | 0.53390165 |
| BRAF | 7 | −140732563 | −140924928 | 0.04879259 | 13.2715857 | −2.2586842 |
| LAMB1 | 7 | −107923800 | −108003359 | 0.06300422 | 17.1371481 | 0.81293874 |
| CDK6 | 7 | −92604920 | −92833950 | 0.04594812 | 12.4978882 | −0.7545512 |
| ABCB1 | 7 | −87503862 | −87600888 | 0.6455159 | 175.580325 | 0.08148807 |
| GUSB | 7 | −65960685 | −65982314 | 0.07288281 | 19.8241254 | 1.47741098 |
| IGFBP3 | 7 | −45912244 | −45921272 | 0.16504814 | 44.8930953 | 0.28777414 |
| SFRP4 | 7 | −37905932 | −37916923 | 0.07381845 | 20.0786175 | 0.28145985 |
| ETV1 | 7 | −13891230 | −13990702 | 0.02748404 | 7.47565756 | 0.91741522 |
| PDGFA | 7 | −497259 | −519844 | 0.40196003 | 109.333127 | −0.5331505 |
| IL6 | 7 | 22727141 | 22732002 | 0.99676218 | 271.119312 | −0.0015857 |
| EGFR | 7 | 55019020 | 55208080 | 0.12494372 | 33.9846927 | 0.96375967 |
| SERPINE1 | 7 | 101127088 | 101139266 | 0.58060144 | 157.923591 | −0.1482684 |
| CAV1 | 7 | 116524784 | 116561185 | 0.91458624 | 248.767458 | 0.03031777 |
| MET | 7 | 116672358 | 116798386 | 0.97314151 | 264.694492 | 0.00905717 |
| SMO | 7 | 129188871 | 129213544 | 0.14807786 | 40.2771775 | 1.1469522 |
| CASP2 | 7 | 143288214 | 143307696 | 0.22786242 | 61.9785791 | 1.01979666 |
| PLAT | 8 | −42174717 | −42207724 | 0.47157657 | 128.268827 | −0.2286388 |
| SFRP1 | 8 | −41261956 | −41309471 | 0.62300936 | 169.458546 | −0.0699084 |
| FGFR1 | 8 | −38411137 | −38468834 | 0.61790641 | 168.070545 | 0.26832816 |
| TNFRSF10B | 8 | −23020135 | −23069187 | 0.74292093 | 202.074492 | −0.1642084 |
| DLC1 | 8 | −13304606 | −13514920 | 0.03167163 | 8.61468211 | −0.8460009 |
| LYN | 8 | 55879826 | 56012447 | 0.38373517 | 104.375967 | −0.617398 |
| MYC | 8 | 127735433 | 127742951 | 0.06640466 | 18.0620676 | 0.76173925 |
| NOTCH1 | 9 | −136494432 | −136545786 | 0.93021333 | 253.018027 | 0.05531615 |
| KLF4 | 9 | −107484856 | −107489720 | 0.53605703 | 145.807513 | −0.2143036 |
| GAS1 | 9 | −86944361 | −86947189 | 0.1763952 | 47.9794949 | −0.5159676 |
| FANCG | 9 | −35073837 | −35080016 | 0.30646399 | 83.3582058 | 0.68784205 |
| CDKN2B | 9 | −22002902 | −22009313 | 0.27384403 | 74.4855761 | 0.50885909 |
| CDKN2A | 9 | −21967751 | −21994491 | 0.64813716 | 176.293309 | 0.25718432 |
| TEK | 9 | 27109140 | 27230178 | 0.05767108 | 15.6865348 | −0.6471249 |
| NTRK2 | 9 | 84668457 | 85027070 | 0.15425707 | 41.9579222 | −0.2835173 |
| DAPK1 | 9 | 87497227 | 87708634 | 0.03072929 | 8.35836663 | −1.0335753 |
| CKS2 | 9 | 89311194 | 89316703 | 6.1882E−05 | 0.01683203 | 1.81806458 |
| SYK | 9 | 90801679 | 90898560 | 0.03907185 | 10.6275441 | −1.0342368 |
| ABL1 | 9 | 130713880 | 130887675 | 0.98278569 | 267.317708 | −0.0166264 |
| FGFR2 | 10 | −121481852 | −121593967 | 0.3376725 | 91.8469197 | −0.2514618 |
| ITGB1 | 10 | −32900317 | −32935558 | 0.81474577 | 221.610849 | 0.21368819 |
| BMI1 | 10 | 22321209 | 22331485 | 0.00065957 | 0.1794029 | −2.2249155 |
| MAP3K8 | 10 | 30434020 | 30461833 | 0.1012968 | 27.5527298 | −0.9652761 |
| RET | 10 | 43077026 | 43130349 | 0.52813917 | 143.653853 | 0.28642217 |
| CDC2 | 10 | 60778331 | 60794852 | 0.00013636 | 0.03708869 | 1.34397181 |
| PTEN | 10 | 87863437 | 87971930 | 0.02926554 | 7.96022724 | −1.7150518 |
| FAS | 10 | 88990558 | 89017061 | 0.06142184 | 16.7067408 | −1.2293204 |
| ETS1 | 11 | −128458760 | −128522401 | 0.8668429 | 235.781269 | −0.0901599 |
| MMP3 | 11 | −102835796 | −102843689 | 0.33895132 | 92.1947591 | 0.58277529 |
| MMP1 | 11 | −102789909 | −102798235 | 0.92234349 | 250.877428 | 0.06105644 |
| PGR | 11 | −101029623 | −101129813 | 0.00985845 | 2.68149744 | −0.5912574 |
| NUMA1 | 11 | −72002863 | −72080474 | 0.09051434 | 24.6199004 | −1.7888464 |
| SPI1 | 11 | −47354857 | −47378576 | 0.37686593 | 102.507533 | −0.4574709 |
| LMO2 | 11 | −33858575 | −33892289 | 0.66625177 | 181.220482 | −0.2276128 |
| WT1 | 11 | −32387775 | −32435535 | 0.51485998 | 140.041914 | 0.44966097 |
| LMO1 | 11 | −8224303 | −8268635 | 0.12989709 | 35.3320082 | −0.7838592 |
| IGF2 | 11 | −2129111 | −2138974 | 0.04241165 | 11.5359687 | 0.40260946 |
| HRAS | 11 | −532241 | −535567 | 0.63626477 | 173.064016 | 0.41963104 |
| RRM1 | 11 | 4094684 | 4138925 | 0.00261434 | 0.71110044 | 2.35267155 |
| WEE1 | 11 | 9573680 | 9589766 | 0.84822318 | 230.716705 | −0.1634172 |
| CD44 | 11 | 35138869 | 35232402 | 0.21299331 | 57.9341815 | −0.4026181 |
| CCND1 | 11 | 69641104 | 69654474 | 0.00023993 | 0.06526052 | 1.79395978 |
| FOLR1 | 11 | 72189557 | 72196323 | 0.07437003 | 20.2286472 | −0.7100955 |
| BIRC2 | 11 | 102347181 | 102378670 | 0.23833139 | 64.8261384 | −1.1748906 |
| ATM | 11 | 108222831 | 108229364 | 0.64951043 | 176.666838 | −0.454259 |
| MLL | 11 | 118436490 | 118526832 | 0.2933901 | 79.8021076 | −1.4712117 |
| CHEK1 | 11 | 125625135 | 125657147 | 0.00061988 | 0.16860613 | 1.8066039 |
| IGF1 | 12 | −102417493 | −102480645 | 0.48646112 | 132.317426 | 0.20138848 |
| CDK4 | 12 | −57747726 | −57752447 | 0.02231324 | 6.06920113 | 2.01421604 |
| WNT10B | 12 | −48965339 | −48971858 | 0.33489171 | 91.0905462 | 0.32560684 |
| PTHLH | 12 | −27962320 | −27971983 | 0.51377409 | 139.746554 | −0.1719157 |
| KRAS | 12 | −25204788 | −25250931 | 0.06212446 | 16.8978531 | −1.5881557 |
| EPS8 | 12 | −15620140 | −15789576 | 0.21130869 | 57.4759642 | −0.6204421 |
| FOXM1 | 12 | −2857680 | −2877155 | 3.3423E−05 | 0.00909119 | 1.33894131 |
| CCND2 | 12 | 4273735 | 4305356 | 0.01366493 | 3.71686231 | −0.5585121 |
| GAPDH | 12 | 6534404 | 6538375 | 0.02431312 | 6.61316787 | 1.42957129 |
| ETV6 | 12 | 11649853 | 11895391 | 0.18592546 | 50.5717246 | −1.0625616 |
| WNT1 | 12 | 48978452 | 48982613 | 0.04718206 | 12.8335205 | −1.5519506 |
| IGFBP6 | 12 | 53097651 | 53102344 | 0.07353706 | 20.0020797 | 0.44700407 |
| CDK2 | 12 | 55966768 | 55972789 | 0.95006002 | 258.416326 | 0.04880993 |
| ERBB3 | 12 | 56080024 | 56103507 | 0.78582176 | 213.74352 | −0.0803922 |
| PTPN11 | 12 | 112418897 | 112486923 | 0.20143908 | 54.7914296 | 1.24276352 |
| FLT1 | 13 | −28399043 | −28495128 | 0.00320648 | 0.87216209 | −1.6477836 |
| FLT3 | 13 | −28003273 | −28100592 | 0.70170603 | 190.86404 | −0.1963633 |
| BRCA2 | 13 | 32315479 | 32399672 | 0.00078694 | 0.21404721 | 1.8104941 |
| RB1 | 13 | 48303746 | 48481890 | 0.31647583 | 86.0814262 | −0.9014911 |
| TFDP1 | 13 | 113584687 | 113641473 | 0.40601477 | 110.436017 | 0.95821477 |
| AKT1 | 14 | −104769349 | −104795743 | 0.94608218 | 257.334352 | 0.04510563 |
| FOXN3 | 14 | −89156171 | −89417110 | 0.57311588 | 155.887518 | −0.4049564 |
| LTBP2 | 14 | −74498182 | −74612331 | 0.01465747 | 3.98683195 | −0.5444944 |
| TMEM30B | 14 | −61277370 | −61281812 | 1.8167E−05 | 0.00494132 | −0.7588811 |
| BMP4 | 14 | −53949735 | −53955050 | 0.03216955 | 8.75011855 | −0.6258317 |
| NDRG2 | 14 | −21016762 | −21025776 | 0.0012002 | 0.32645319 | −1.6190995 |
| MMP14 | 14 | 22836532 | 22847599 | 0.09016387 | 24.5245731 | 0.84683901 |
| HIF1A | 14 | 61695400 | 61748259 | 0.56397898 | 153.402281 | −0.4141385 |
| FOS | 14 | 75278777 | 75282234 | 0.60464254 | 164.46277 | −0.158795 |
| YY1 | 14 | 100238764 | 100279034 | 0.90828286 | 247.052937 | −0.119839 |
| MEG3 | 14 | 100826107 | 100861023 | 0.84594569 | 230.097228 | −0.0334485 |
| MTA1 | 14 | 105419848 | 105470720 | 0.91837935 | 249.799184 | −0.0783046 |
| NTRK3 | 15 | −87977364 | −88256731 | 0.22104373 | 60.1238935 | −0.4141425 |
| BCL2A1 | 15 | −79960889 | −79971301 | 0.04852125 | 13.1977797 | 0.96575802 |
| CYP1A1 | 15 | −74719541 | −74725610 | 0.81549547 | 221.814769 | 0.12063228 |
| TYRO3 | 15 | 41559021 | 41579338 | 0.15158098 | 41.2300266 | 0.73170813 |
| PML | 15 | 73994672 | 74043376 | 0.76685869 | 208.585562 | 0.22788447 |
| CSK | 15 | 74782083 | 74803198 | 0.61072889 | 166.118259 | 0.47817955 |
| BLM | 15 | 90717326 | 90815462 | 0.00082056 | 0.22319321 | 1.77050293 |
| NQO1 | 16 | −69709400 | −69726630 | 0.00213481 | 0.58066896 | 1.25565365 |
| CDH11 | 16 | −64943752 | −65122137 | 0.07672077 | 20.8680491 | 0.89878163 |
| SIAH1 | 16 | −48360542 | −48385318 | 0.25700055 | 69.9041506 | −0.920784 |
| TRAF7 | 16 | 2155797 | 2178129 | 0.94564577 | 257.215648 | −0.0672052 |
| ERCC4 | 16 | 13920156 | 13952348 | 0.02221116 | 6.04143471 | −2.1891985 |
| MMP2 | 16 | 55478829 | 55506691 | 0.15662356 | 42.6016091 | −0.4067978 |
| CDH1 | 16 | 68737289 | 68835542 | 0.26842353 | 73.0111995 | 0.38476364 |
| TIMP2 | 17 | −78852976 | −78925390 | 0.14298441 | 38.8917585 | 0.91608905 |
| COL1A1 | 17 | −50184095 | −50201639 | 0.01666466 | 4.53278745 | 0.53683636 |
| BRCA1 | 17 | −43044294 | −43125451 | 0.03787326 | 10.3015277 | 1.32866726 |
| STAT3 | 17 | −42313324 | −42388387 | 0.00259212 | 0.70505595 | −2.6968533 |
| SMARCE1 | 17 | −40627727 | −40647851 | 0.10566341 | 28.7404482 | 1.57188639 |
| TOP2A | 17 | −40388520 | −40417950 | 5.9183E−05 | 0.01609769 | 1.31951539 |
| TP53 | 17 | −7668401 | −7675493 | 0.10053225 | 27.3447713 | 1.11484577 |
| SERPINF1 | 17 | 1761924 | 1777574 | 0.08365138 | 22.7531766 | −0.3264401 |
| NF1 | 17 | 31094926 | 31222764 | 0.1029693 | 28.0076489 | −1.7762071 |
| CDK5R1 | 17 | 32487086 | 32491253 | 0.38849525 | 105.670709 | 0.43359314 |
| ERBB2 | 17 | 39688083 | 39728662 | 0.81733809 | 222.315961 | −0.1737569 |
| GRB7 | 17 | 39737900 | 39747285 | 0.6518605 | 177.306057 | 0.25505935 |
| CSF3 | 17 | 40015360 | 40017813 | 0.32265466 | 87.7620687 | −0.6447777 |
| RARA | 17 | 40309170 | 40357643 | 0.41892742 | 113.948258 | 0.71312509 |
| NGFR | 17 | 49495292 | 49515020 | 0.35402243 | 96.2940999 | 0.3504463 |
| CLTC | 17 | 59619688 | 59696956 | 0.00076857 | 0.20905223 | 2.03589946 |
| PRKAR1A | 17 | 68413622 | 68533429 | 0.5781366 | 157.253155 | −0.5174001 |
| BIRC5 | 17 | 78214195 | 78225635 | 1.6139E−05 | 0.00438991 | 1.42351428 |
| BCL2 | 18 | −63317953 | −63319380 | 0.03638934 | 9.89790004 | −0.9308328 |
| YES1 | 18 | −721591 | −812326 | 0.03592291 | 9.77103078 | −1.4971859 |
| TYMS | 18 | 657583 | 673577 | 0.12669475 | 34.4609728 | 0.74783071 |
| ERCC2 | 19 | −45358760 | −45370587 | 0.94157259 | 256.107744 | 0.05282785 |
| PLAUR | 19 | −43648570 | −43670346 | 0.01963791 | 5.34151279 | 1.13571773 |
| TGFB1 | 19 | −41330322 | −41353883 | 0.66755787 | 181.575741 | −0.27194 |
| AKT2 | 19 | −40230316 | −40285395 | 0.09668037 | 26.2970595 | 2.02632352 |
| CEBPA | 19 | −33299933 | −33302564 | 0.6182034 | 168.151324 | −0.1852666 |
| JUNB | 19 | 12791495 | 12793311 | 0.7476102 | 203.349974 | −0.1030817 |
| CCNE1 | 19 | 29811993 | 29824317 | 0.33961987 | 92.3766035 | 0.53206043 |
| BCL3 | 19 | 44748720 | 44760044 | 0.12911451 | 35.1191475 | 0.60941008 |
| E2F1 | 20 | −33675485 | −33686404 | 0.0042748 | 1.1627445 | 1.31590672 |
| BCL2L1 | 20 | −31664457 | −31723098 | 0.13727011 | 37.3374703 | 0.79587429 |
| PCNA | 20 | −5114952 | −5126622 | 0.00165707 | 0.4507222 | 2.25177197 |
| CDC25B | 20 | 3786771 | 3806121 | 0.01497635 | 4.07356824 | 1.12542267 |
| HCK | 20 | 32052187 | 32101854 | 0.36341832 | 98.8497827 | −0.4707421 |
| TOP1 | 20 | 41028817 | 41124486 | 0.0674009 | 18.3330452 | 1.39863769 |
| MYBL2 | 20 | 43667018 | 43716496 | 2.7568E−06 | 0.00074984 | 1.51155495 |
| WFDC2 | 20 | 45469753 | 45481532 | 0.99892631 | 271.707957 | 0.00031258 |
| MMP9 | 20 | 46008907 | 46016561 | 0.36065657 | 98.098586 | 0.24191698 |
| GNAS | 20 | 58839717 | 58911196 | 0.04195898 | 11.4128436 | −1.1682863 |
| SOD1 | 21 | 31659621 | 31668930 | 0.54822327 | 149.11673 | 0.44002217 |
| ETS2 | 21 | 38805306 | 38824954 | 0.59029116 | 160.559195 | −0.4013471 |
| LIF | 22 | −30240446 | −30246851 | 0.70891111 | 192.823823 | 0.17774294 |
| AP1B1 | 22 | −29327679 | −29388583 | 0.25552486 | 69.5027611 | −0.9061868 |
| BCR | 22 | 23180364 | 23318037 | 0.42358733 | 115.215753 | −0.475879 |
| NF2 | 22 | 29603555 | 29698600 | 0.02572348 | 6.99678764 | −1.1508729 |
| TIMP3 | 22 | 32800815 | 32863041 | 0.04208841 | 11.4480478 | −0.6124857 |
| L1CAM | X | −153861513 | −153886174 | 0.00802368 | 2.18244164 | 0.58796089 |
| TFE3 | X | −49028725 | −49043517 | 0.33878458 | 92.1494058 | −0.9389898 |
| TIMP1 | X | 47582290 | 47586791 | 0.02099598 | 5.71090618 | 1.19969111 |
| GATA1 | X | 48786573 | 48794310 | 0.54253189 | 147.568673 | −0.2970761 |
| AR | X | 67544622 | 67730619 | 0.09290562 | 25.2703279 | −0.3956902 |
| PGK1 | X | 78104168 | 78126827 | 0.00505158 | 1.37403039 | 1.72142595 |
| HPRT1 | X | 134460144 | 134500668 | 0.02883323 | 7.8426374 | 1.80919506 |
| PCTK1 | X | 47217860 | 47229997 | 0.01776539 | 4.83218568 | 2.50698016 |

TABLE 3
Meningioma gene signature discovery and validation
cohort characteristics for Example 2.

| | Discovery | | Validation | |
|---|---|---|---|---|
| Meningiomas - no. | 173 | | 331 | |
| Patients - no. | 166 | | 294 | |
| Females - no. (%) | 112 | (67.5) | 206 | (70.1) |
| Median age (IQR) - yr. | 57.0 | (45.0-65.1) | 58.1 | (48.8-68.0) |
| Setting - no. (%) | | | | |
| Primary | 143 | (82.7) | 274 | (82.8) |
| Recurrent | 30 | (17.3) | 57 | (17.2) |
| Extent of resection - no. (%) | | | | |
| Gross total | 110 | (63.6) | 255 | (77.0) |
| Subtotal | 63 | (36.4) | 76 | (23.0) |
| WHO Grade - no. (%) | | | | |
| 1 | 83 | (50.0) | 271 | (81.9) |
| 2 | 65 | (37.6) | 55 | (16.6) |
| 3 | 25 | (14.4) | 5 | (1.5) |
| Ch1p loss - no. (%) | 60 | (34.7) | 61 | (18.4) |
| Ch22q loss - no. (%) | 89 | (51.4) | 181 | (54.7) |
| DNA methylation | | | | |
| group - no. (%) | | | | |
| Merlin-intact | 67 | (38.7) | 112 | (33.8) |
| Immune-enriched | 54 | (31.2) | 133 | (40.2) |
| Hypermitotic | 52 | (30.1) | 86 | (26.0) |
| Adjuvant | 33 | (19.1) | 40 | (12.1) |
| radiotherapy - no. (%) | | | | |
| Median follow up (IQR) - yr. | 8.1 | (3.9-11.9) | 6.1 | (2.0-9.7) |
| Local recurrence - no. (%) | 61 | (35.3) | 78 | (23.6) |
| Death - no. (%) | | | | |
| Meningioma | 24 | (13.8) | 28 | (8.5) |
| Other | 11 | (6.4) | 29 | (8.8) |
| Unknown | 11 | (6.4) | 15 | (4.5) |

TABLE 4
Targeted gene expression discovery panel (Example 2)

| Target | Gene | NCBI Standard ID (RefSeq) |
|---|---|---|
| Meningioma | APEX2 | NM_014481.3:1990 |
| Meningioma | AREG | NM_001657.2:547 |
| Meningioma | ARID1B | NM_020732.3:6335 |
| Meningioma | AURKA | NM_003600.2:405 |
| Meningioma | AURKB | NM_004217.2:615 |
| Meningioma | BIRC5 | NM_001168.2:1215 |
| Meningioma | BMI1 | NM_005180.5:1145 |
| Meningioma | BMP4 | NM_001202.3:659 |
| Meningioma | BMPR1A | NM_004329.2:1720 |
| Meningioma | BRIP1 | NM_032043.2:2512 |
| Meningioma | CCL21 | NM_002989.2:180 |
| Meningioma | CCN1 | NM_001554.3:1390 |
| Meningioma | CCN2 | NM_001901.2:1100 |
| Meningioma | CCNA2 | NM_001237.2:1210 |
| Meningioma | CCNB1 | NM_031966.3:1102 |
| Meningioma | CCND2 | NM_001759.2:5825 |
| Meningioma | CCND3 | NM_001760.2:1215 |
| Meningioma | CD3E | NM_000733.2:75 |
| Meningioma | CDC20 | NM_001255.2:430 |
| Meningioma | CDC25A | NM_001789.2:1229 |
| Meningioma | CDC25C | NM_001287582.1:1944 |
| Meningioma | CDK1 | NM_001786.4:178 |
| Meningioma | CDK4 | NM_000075.2:1055 |
| Meningioma | CDK6 | NM_001259.6:2404 |
| Meningioma | CDKN2A | NM_000077.4:559 |
| Meningioma | CDKN2C | NM_001262.2:1295 |
| Meningioma | CDKN3 | NM_005192.3:510 |
| Meningioma | CENPF | NM_016343.3:5822 |
| Meningioma | CHEK1 | NM_001114121.1:2225 |
| Meningioma | CKS2 | NM_001827.1:195 |
| Meningioma | COL1A1 | NM_000088.3:5210 |
| Meningioma | CXCL8 | NM_000584.2:25 |
| Meningioma | DRAM1 | NM_018370.2:1730 |
| Meningioma | DTL | NM_016448.2:715 |
| Meningioma | E2F2 | NM_004091.3:1104 |
| Meningioma | EME1 | XM_011524392.1:416 |
| Meningioma | ERCC4 | NM_005236.2:1700 |
| Meningioma | ESR1 | NM_000125.2:1595 |
| Meningioma | EZH2 | NM_001203247.1:1121 |
| Meningioma | FANCB | NM_001018113.2:238 |
| Meningioma | FBLIM1 | NM_001024215.1:976 |
| Meningioma | FGF18 | NM_003862.1:850 |
| Meningioma | FGFR4 | NM_002011.3:1585 |
| Meningioma | FGR | NM_001042747.1:890 |
| Meningioma | FLT1 | NM_002019.4:530 |
| Meningioma | FOXK2 | NM_004514.3:4220 |
| Meningioma | FOXM1 | NM_202002.1:1000 |
| Meningioma | GAS1 | NM_002048.2:1525 |
| Meningioma | GATA3 | NM_001002295.1:1691 |
| Meningioma | GPHA2 | NM_130769.3:274 |
| Meningioma | IFNGR1 | NM_000416.1:1140 |
| Meningioma | IGF2 | NM_000612.4:765 |
| Meningioma | KDR | NM_002253.2:1420 |
| Meningioma | KIF14 | NM_014875.2:4335 |
| Meningioma | KIF20A | NM_005733.2:1209 |
| Meningioma | KRT14 | NM_000526.4:523 |
| Meningioma | L1CAM | NM_024003.2:3240 |
| Meningioma | LINC02593 | NR_026874.2:685 |
| Meningioma | LYVE1 | NM_006691.3:1422 |
| Meningioma | MCM4 | NM_182746.1:1200 |
| Meningioma | MCM6 | NM_005915.4:515 |
| Meningioma | MDM4 | NM_001204172.1:346 |
| Meningioma | MKI67 | NM_002417.2:4020 |
| Meningioma | MMP9 | NM_004994.2:1530 |
| Meningioma | MPL | NM_005373.2:895 |
| Meningioma | MUTYH | NM_001293196.1:1288 |
| Meningioma | MYBL1 | NM_001080416.3:1030 |
| Meningioma | MYBL2 | NM_002466.2:445 |
| Meningioma | NDRG2 | NM_016250.2:1515 |
| Meningioma | NF2 | NM_000268.3:1895 |
| Meningioma | NOTCH2 | NM_024408.3:2842 |
| Meningioma | NOTCH3 | NM_000435.2:1965 |
| Meningioma | NQO1 | NM_000903.2:790 |
| Meningioma | NRAS | NM_002524.3:877 |
| Meningioma | PGK1 | NM_000291.2:1030 |
| Meningioma | PGR | NM_000926.4:2392 |
| Meningioma | PIM1 | NM_002648.2:1630 |
| Meningioma | PINK1 | NM_032409.2:1610 |
| Meningioma | PLAUR | NM_001005376.2:538 |
| Meningioma | PTEN | NM_000314.3:1675 |
| Meningioma | PTPRK | NM_001135648.1:472 |
| Meningioma | PTTG1 | NM_004219.2:542 |
| Meningioma | RACGAP1 | NM_013277.3:1850 |
| Meningioma | RAD51 | NM_133487.2:566 |
| Meningioma | RAD51AP1 | NM_001130862.1:1125 |
| Meningioma | RAD51C | NM_002876.2:300 |
| Meningioma | REL | NM_002908.3:2067 |
| Meningioma | SAMD11 | NM_152486.2:280 |
| Meningioma | SFRP4 | NM_003014.2:1060 |
| Meningioma | SLC7A8 | NM_001267036.1:2662 |
| Meningioma | SMARCE1 | NM_003079.4:690 |
| Meningioma | SOS2 | NM_006939.2:3845 |
| Meningioma | SPOP | NM_001007226.1:1400 |
| Meningioma | SPP1 | NM_000582.2:760 |
| Meningioma | TAGLN | NM_003186.3:260 |
| Meningioma | TERT | NM_198253.1:2570 |
| Meningioma | TMEM30B | NM_001017970.2:2420 |
| Meningioma | TOP2A | NM_001067.3:3563 |
| Meningioma | TRIM37 | NM_015294.4:1910 |
| Meningioma | TROAP | NM_001100620.2:127 |
| Meningioma | USF1 | NM_007122.3:1516 |
| Housekeeping | ACTB | NM_001101.2:1010 |
| Housekeeping | AK3 | NM_016282.2:450 |
| Housekeeping | B2M | NM_004048.2:235 |
| Housekeeping | CASC3 | NM_007359.3:3505 |
| Housekeeping | COQ4 | NM_016035.3:114 |
| Housekeeping | GUSB | NM_000181.3:1899 |
| Housekeeping | HPRT1 | NM_000194.1:240 |
| Housekeeping | LDHA | NM_001165414.1:1690 |
| Housekeeping | LNPK | NM_030650.1:1144 |
| Housekeeping | MRPL19 | NM_014763.3:364 |
| Housekeeping | NUDT2 | NM_147173.1:445 |
| Housekeeping | POP4 | NM_006627.1:765 |
| Housekeeping | PPIA | NM_021130.3:315 |
| Housekeeping | RAPGEF1 | NM_005312.2:1705 |
| Housekeeping | RETREG2 | NM_024293.4:2341 |
| Housekeeping | RPL19 | NM_000981.3:315 |
| Housekeeping | RPLP0 | NM_001002.3:250 |
| Housekeeping | SCRN3 | NM_001193528.1:564 |
| Housekeeping | SDHA | NM_004168.1:230 |
| Housekeeping | SMC5 | NM_015110.3:236 |
| Housekeeping | TTC21B | NM_024753.4:916 |
| Housekeeping | UBQLN1 | NM_053067.2:990 |
| Housekeeping | UXS1 | NM_001253875.1:986 |
| Housekeeping | VCP | NM_007126.2:615 |

TABLE 5
Targeted gene discovery panel, beta_coefficient,
p-value univariate Cox analysis

| Gene | Beta_coefficient | p-value, univariate Cox |
|---|---|---|
| APEX2 | 0.001485584 | 0.5933513 |
| AREG | 0.002247923 | 0.05806676 |
| ARID1B | −0.001121971 | 0.000278304 |
| AURKA | 0.011749121 | 2.7157E−12 |
| AURKB | 0.005113967 | 8.37117E−17 |
| BIRC5 | 0.000810136 | 4.40641E−10 |
| BMI1 | −0.000613721 | 0.022373541 |
| BMP4 | −0.000153278 | 0.000345527 |
| BMPR1A | −0.000289591 | 0.007952081 |
| BRIP1 | 0.005475453 | 6.20496E−11 |
| CCL21 | −0.032695696 | 0.040910507 |
| CCN1 | 1.91388E−05 | 0.097000275 |
| CCN2 | −4.49058E−06 | 0.208701474 |
| CCNA2 | 0.001547103 | 8.71446E−09 |
| CCNB1 | 0.000821751 | 3.589E−09 |
| CCND2 | −0.000103001 | 0.025157444 |
| CCND3 | −0.000869613 | 0.001365172 |
| CD3E | −0.00062843 | 0.779078057 |
| CDC20 | 0.000986475 | 0.056455791 |
| CDC25A | 0.004063081 | 2.65694E−10 |
| CDC25C | 0.003415795 | 9.77053E−05 |
| CDK1 | 0.000914955 | 6.07424E−11 |
| CDK4 | 0.001179153 | 0.012149717 |
| CDK6 | −9.45161E−05 | 0.826513339 |
| CDKN2A | 0.002526386 | 0.000247193 |
| CDKN2C | −0.000150335 | 0.284336373 |
| CDKN3 | 0.000742628 | 0.000179343 |
| CENPF | 0.001359994 | 7.65755E−13 |
| CHEK1 | 0.004561466 | 4.02529E−18 |
| CKS2 | 0.001409011 | 3.37091E−20 |
| COL1A1 | 3.50342E−05 | 3.97985E−06 |
| CXCL8 | 0.000161792 | 1.45903E−07 |
| DRAM1 | −6.71305E−05 | 0.75993945 |
| DTL | 0.007599092 | 2.66478E−11 |
| E2F2 | 0.005997863 | 1.03221E−06 |
| EME1 | 0.006537636 | 5.69538E−11 |
| ERCC4 | −0.000338628 | 0.719887127 |
| ESR1 | 0.000236695 | 0.898804459 |
| EZH2 | 0.005553993 | 8.34608E−12 |
| FANCB | 0.008528613 | 4.09087E−11 |
| FBLIM1 | −0.001176628 | 0.211891831 |
| FGF18 | 0.000946148 | 0.145049773 |
| FGFR4 | 0.000414137 | 0.579675333 |
| FGR | −0.002392588 | 0.07763377 |
| FLT1 | −0.000276836 | 0.08554289 |
| FOXK2 | 0.002145405 | 5.75134E−05 |
| FOXM1 | 0.003915535 | 2.48124E−11 |
| GAS1 | −7.07792E−05 | 0.259673468 |
| GATA3 | 0.000320399 | 0.670671397 |
| GPHA2 | −0.002184305 | 0.319319309 |
| IFNGR1 | −8.0565E−05 | 0.013427428 |
| IGF2 | 7.68003E-06 | 0.010384704 |
| KDR | −0.000540621 | 0.005637778 |
| KIF14 | 0.004214495 | 2.62383E−08 |
| KIF20A | 0.004677545 | 1.61948E−17 |
| KRT14 | −4.04877E−05 | 0.684830441 |
| L1CAM | 0.000499796 | 0.018591667 |
| LINC02593 | −0.007227835 | 5.96113E−06 |
| LYVE1 | −4.20095E−05 | 0.315746614 |
| MCM4 | 0.000615153 | 1.35626E−10 |
| MCM6 | 0.001663922 | 3.2038E−09 |
| MDM4 | 0.000871608 | 2.79536E−06 |
| MKI67 | 0.001962463 | 1.28104E−06 |
| MMP9 | 4.02578E−05 | 0.149823356 |
| MPL | 0.02722747 | 0.014485415 |
| MUTYH | −0.012113783 | 5.10134E−06 |
| MYBL1 | 0.004275401 | 1.96064E−05 |
| MYBL2 | 0.001761497 | 6.79844E−14 |
| NDRG2 | 1.1411E−05 | 0.819770557 |
| NF2 . . . 72 | −0.000777915 | 0.000755103 |
| NOTCH2 | 1.30494E−05 | 0.883500738 |
| NOTCH3 | 0.000287756 | 3.17645E−06 |
| NQO1 | 0.00016044 | 0.018424035 |
| NRAS | 0.001351824 | 0.003192726 |
| PGK1 | 0.00010351 | 9.35481E−14 |
| PGR | −0.000239554 | 0.133772415 |
| PIM1 | 0.00069876 | 7.5013E−15 |
| PINK1 | −0.00080038 | 5.43223E−06 |
| PLAUR | 0.002181596 | 1.41001E−06 |
| PTEN | −0.000423136 | 3.14631E−05 |
| PTPRK | −0.000375872 | 0.578637365 |
| PTTG1 | 0.000415804 | 8.99573E−07 |
| RACGAP1 | 0.000316582 | 0.002109952 |
| RAD51 | 0.006418272 | 3.2676E−09 |
| RAD51AP1 | 0.002941767 | 8.12335E−10 |
| RAD51C | 0.001329825 | 2.61868E−06 |
| REL | 0.000134244 | 0.764299311 |
| SAMD11 | −0.001612901 | 1.17045E−08 |
| SFRP4 | 2.12162E−05 | 0.620484554 |
| SLC7A8 | −0.000231729 | 0.290577842 |
| SMARCE1 | 1.43106E−05 | 0.86201186 |
| SOS2 | −0.000785304 | 0.00131929 |
| SPOP | −0.000566071 | 0.064157284 |
| SPP1 | 2.78128E−05 | 7.58963E−07 |
| TAGLN | 2.53135E−05 | 0.038461805 |
| TERT | 0.009956771 | 4.78817E−07 |
| TMEM30B | −8.51571E−05 | 7.32408E−06 |
| TOP2A | 0.000360698 | 4.21249E−09 |
| TRIM37 | 0.000659631 | 2.49892E−12 |
| TROAP | 0.003161622 | 6.91426E−07 |
| USF1 | 0.00348342 | 0.011339035 |

TABLE 6
Targeted gene expression biomarker panel (Example 2)
(includes genes, rationale, function and references)

| Gene | Rationale/Function | Chr | Band | PMID |
|---|---|---|---|---|
| ARID1B | Chromatin regulation, tumor suppressor | 6 | q25.3 | 28195122; 30202034 |
| CCL21 | Meningeal lymphatics | 9 | p13.3 | Choudhury et al: |
| | | | | www.medrxiv.org/content/10.1 |
| | | | | 101/2020.11.23.20237495v1 |
| CCN1 | Growth factor signaling, pro-proliferative, | 1 | p22.3 | 32860417; 20685720 |
| | preliminary gene expression risk score | | | |
| | component | | | |
| CCND2 | Cell proliferation, preliminary gene | 12 | p13.32 | 32860417; 20386868 |
| | expression risk score component | | | |
| CD3E | Meningeal lymphatics | 11 | q23.3 | Choudhury et al: |
| | | | | www.medrxiv.org/content/10.1 |
| | | | | 101/2020.11.23.20237495v1 |
| CDC20 | Cell proliferation | 1 | p34.2 | 20015288; 28195122; |
| | | | | 24724603 |
| CDK6 | Cell proliferation | 7 | q21.2 | 18048012; 29391485; |
| | | | | 25148008 |
| CDKN2A | Inhibits cell proliferation | 9 | p21.3 | 21168406; 11485924 |
| CDKN2C | Inhibits cell proliferation | 1 | p32.3 | 11485924 |
| CHEK1 | Cell cycle regulation, DNA damage | 11 | q24.2 | 20015288; 21945852 |
| | response | | | |
| CKS2 | Cell proliferation, preliminary gene | 9 | q22.2 | 32860417; 20015288; |
| | expression risk score component | | | 21948653 |
| COL1A1 | Extracellular matrix, invasion and | 17 | q21.33 | 32860417; 23285163; |
| | migration, preliminary gene expression risk | | | 27096627 |
| | score component | | | |
| ESR1 | Estrogen hormone signaling | 6 | q25.1 | 28195122; 25965831 |
| EZH2 | Chromatin and methylation regulation, cell | 7 | q36.1 | 28195122; 32729292; |
| | proliferation | | | 31591222 |
| FBLIM1 | 1p36 marker | 1 | p36.21 | 20015288 |
| FGFR4 | Growth factor signaling | 5 | q35.2 | 20015288; 28552950; |
| | | | | 19918127 |
| GAS1 | Inhibits cell proliferation, preliminary gene | 9 | q21.33 | 32860417; 20386868; |
| | expression risk score component | | | 20015288 |
| IFNGR1 | Cytokine signaling, preliminary gene | 6 | q23.3 | 32860417; 32860417 |
| | expression risk score component | | | |
| IGF2 | IGF2 signaling, preliminary gene | 11 | p15.5 | 32860417; 20386868; |
| | expression risk score component | | | 15540215 |
| KDR | Angiogenesis, preliminary gene expression | 4 | q12 | 30519894; 32860417 |
| | risk score component | | | |
| KIF20A | Mitotic stability, cell proliferation | 5 | q31.2 | 21168406; 30991738 |
| KRT14 | Invasion and migration | 17 | q21.2 | 21168406 |
| LINC02593 | 1p36 marker | 1 | p36.33 | 21168406 |
| MDM4 | Oncogene, cell proliferation regulator | 1 | q32.1 | 21168406; 15540215 |
| MMP9 | Extracellular matrix remodeling, invasion, | 20 | q13.12 | 32860417; 20652360; |
| | preliminary gene expression risk score | | | 11702875; 25821815 |
| | component | | | |
| MUTYH | DNA damage repair | 1 | p34.1 | 28195122; 20150366 |
| MYBL1 | Cell proliferation, cell cycle regulation | 8 | q13.1 | 32860417 |
| PGK1 | Glycolysis metabolism | X | q21.1 | 20015288 |
| PGR | Progesterone hormone signaling, | 11 | q22.1 | 32860417; 15452155; |
| | preliminary gene expression risk score | | | 8443810; 8988089 |
| | component | | | |
| PIM1 | Oncogene, preliminary gene expression | 6 | p21.2 | 32860417; 21318223 |
| | risk score component | | | |
| SPOP | Tumor suppressor, ubiquination regulation | 17 | q21.33 | 20015288 |
| TAGLN | Cytoskeletal organization, invasion and | 11 | q23.3 | 28195122; 29424888; |
| | migration | | | 15540215; 24289128 |
| TMEM30B | Preliminary gene expression risk score | 14 | q23.1 | 32860417; 20685720 |
| | component | | | |
| USF1 | Cell proliferation, transcription factor | 1 | q23.3 | Choudhury et al: |
| | | | | www.medrxiv.org/content/10.1 |
| | | | | 101/2020.11.23.20237495v1 |
| ACTB | Housekeeping | 7 | p22.1 | 21806841 |
| CASC3 | Housekeeping | 17 | q21.1 | 21806841 |
| GUSB | Housekeeping | 7 | q11.21 | 21806841 |
| KIAA1715 | Housekeeping | 2 | q31.1 | 17878933 |
| MRPL19 | Housekeeping | 2 | p12 | 17878933 |
| POP4 | Housekeeping | 19 | q12 | 17878933 |
| TTC21B | Housekeeping | 2 | q24.3 | 17878933 |

What is claimed is:

1. A method of evaluating the likelihood of recurrence of meningioma in a patient, the method comprising:
detecting the levels of expression of each member of a panel of 36 genes or a panel that comprises a subset of at least six genes of the 36-gene panel, in a sample from the patient that comprises meningioma tumor cells, wherein the 36 genes are: FRP, NRAS, NQO1, COL1A1, CDC25C, MYBL2, CDC2, FOXM1, BIRC5, TOP2A, L1CAM MMP9, SPP1, CXCL8, PIM1, PLAUR, IGF2, FLT1, KDR, AREG, NF2, FGR, CCND3, NDRG2, ERCC4, CCND2, BMI1, REL, MPL, BMP4, CYR61, CTGF, GAS1, IFNGR1, TMEM30B, and PGR;
determining a normalized value for the level of expression of each member of the panel and assigning an expression score to each normalized value;
summing the expression score for each gene to assign a risk score for the likelihood of recurrence of meningioma.

2. The method of claim 1, wherein a high risk score in the top third tertile compared to a reference scale is indicative of a high risk of local recurrence.

3. The method of claim 1 or 2, wherein the subset comprise at least two genes from each of the following subgroups: Group 1, SFRP4, NRAS, NQO1, COL1A1, CDC25C, MYBL2, CDC2/CDK1, FOXM1, BIRC5, and TOP2A; Group 2, L1CAM, MMP9, SPP1, CXCL8, PIM1, PLAUR, and IGF2; Group 3, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR.

4. The method of claim 3, wherein the subset comprises at least three genes, or at least four genes, from each of the subgroups.

5. The method of claim 1 or 2, wherein the subset comprises at least one gene that is localized to chromosome arm 1p, at least one gene that is localized to chromosome arm 1q, at least one gene that is localized to chromosome arm 6q, at least one gene that is localized to chromosome arm 17q, and at least one gene that is localized to chromosome arm 20q.

6. The method of claim 5, wherein the subset further comprises at least one gene that is localized to chromosome arm 3p, at least one gene that is localized to chromosome arm 7q, at least one gene that is localized to chromosome arm 11q, at least one gene that is localized to chromosome arm 14q, and at least one gene that is localized to chromosome arm 22q.

7. The method of any one of claims 1 to 6, wherein expression is detected by determining levels of RNA transcripts encoded by the genes.

8. The method of claim 7, determining the level of the RNA transcripts comprises performing an amplification assay, a hybridization assay, a sequencing assay or an array-based hybridization assay.

9. The method of claim 1 or 2, further comprising recommending radiotherapy treatment to the patient when the patient has a high risk score.

10. The method of any one of claims 1 to 6, wherein expression is detected by determining levels of proteins encoded by the genes.

11. The method of claim 10, wherein detecting the level of protein comprises performing an immunoassay.

12. The method of any one of claims 1 to 11, wherein the reference scale is a plurality of risk scores derived from a population of reference patients that have meningioma.

13. The method of any one of claims 1 to 12, wherein the sample from the patient is a tumor tissue sample or a tumor cell sample.

14. A microarray comprising probes for detecting expression of a gene panel for predicting survival, wherein the gene panel consists of the gene FRP, NRAS, NQO1, COL1A1, CDC25C, MYBL2, CDC2, FOXM1, BIRC5, TOP2A, L1CAM, MMP9, SPP1, CXCL8, PIM1 PLAUR, IGF2, FLT1, KDR, AREG, NF2, FGR, CCND3, NDRG2, ERCC4, CCND2, BMI1, REL, MPL, BMP4, CYR61, CTGF, GAS1, IFNGR1, TMEM30B, and PGR, or a subset of at least 6 genes of the gene panel; and optionally contains probes for detecting expression of one or more reference genes, wherein the microarray contains probes for detecting no more than 1,000 gene, nor more than 500 genes, nor more than 200 genes, or no more than 100 genes.

15. The microarray of claim 14, wherein the subset comprise at least two genes from each of the following subgroups: Group 1, SFRP4, NRAS, NQO1, COL1A1, CDC25C, MYBL2, CDC2/CDK1, FOXM1, BIRC5, and TOP2A; Group 2, L1CAM MMP1, SPP1, CXCL8, PIM1, PLAUR, and IGF2; Group 3, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR.

16. The microarray of claim 15, wherein the subset comprises at least three genes, or at least four genes, from each of the subgroups.

17. The microarray of claim 14, wherein the subset comprises at least one gene that is localized to chromosome arm 1p, at least one gene that is localized to chromosome arm 1q, at least one gene that is localized to chromosome arm 6q, at least one gene that is localized to chromosome arm 17q, and at least one gene that is localized to chromosome arm 20q.

18. The microarray of claim 17, wherein the subset further comprises at least one gene that is localized to chromosome arm 3p, at least one gene that is localized to chromosome arm 7q, at least one gene that is localized to chromosome arm 11q, at least one gene that is localized to chromosome arm 14q, and at least one gene that is localized to chromosome arm 22q.

19. A kit comprising primers and/or probes for detecting expression of a gene panel for predicting survival, wherein the gene panel consists of the gene SFRP, NRAS, NQO1, COL1A1, CDC25C, MYBL2, CDC2, FOXM1, BIRC5, TOP2A, L1CAM, MMP9, SPP1, CXCL8, PIM1, PLAUR, IGF2, FLT1, KDR, AREG, NF2, FGR, CCND3, NDRG2, ERCC4, CCND2, BMI1, REL, MPL, BMP4, CYR61, CTGF, GAS1, IFNGR1, TMEM30B, and PGR, or a subset of at least 6 genes of the gene panel, and optionally contains primers and/or probes for detecting expression of one or more reference genes.

20. The kit of claim 19, wherein the subset comprise at least two genes from each of the following subgroups: Group 1, SFRP4, NRAS, NQO1, COL1A1, CDC25C, MYBL2, CDC2/CDK1, FOXM1, BIRC5, and TOP2A; Group 2, L1CAM, MMP9, SPP1, CXCL8, PIM1, PLAUR, and IGF2; Group 3, FLT1, KDR, AREG, NF2, FGR, CCND3, NDRG2, ERCC4, CCND2, BMI1, REL, MPL, BMP4, CYR61/CCN1, CTGF/CCN2, GAS1, IFNGR1, TMEM30B, and PGR.

21. The kit of claim 20, wherein the subset comprises at least three genes, or at least four genes, from each of the subgroups.

22. The kit of claim 19, wherein the subset comprises at least one gene that is localized to chromosome arm 1p, at least one gene that is localized to chromosome arm 1q, at least one gene that is localized to chromosome arm 6q, at least one gene that is localized to chromosome arm 17q, and at least one gene that is localized to chromosome arm 20q.

23. The kit of claim of 22, wherein the subset further comprises at least one gene that is localized to chromosome arm 3p, at least one gene that is localized to chromosome arm 7q, at least one gene that is localized to chromosome arm 11q, at least one gene that is localized to chromosome arm 14q, and at least one gene that is localized to chromosome arm 22q

24. A method of evaluating the likelihood of recurrence of meningioma in a patient, the method comprising:
detecting the levels of expression of each member of a panel of 34 genes or a panel that comprises a subset of at least eight genes of the 34-gene panel, in a sample from the patient that comprises meningioma tumor cells, wherein the 34 genes are: ARID1B, CCL21, CCN1, CCND2, CD3E, CDC20, CDK6, CDKN2A, CDKN2C, CHEK1, CKS2, COL1A1, ESR1, EZH2, FBLIM1, FGFR4, GAS1, IFNGR1, IGF2, KDR, KIF20A, KRT14, LINC02593, MDM4, MMP9. MUTYH, MYBL1, PGK1. PGR, PIM1, SPOP. TAGLN, TMEM30B, and USF1;
determining a normalized value for the level of expression of each member of the panel and assigning an expression score to each normalized value;
summing the expression score for each gene to assign a risk score for the likelihood of recurrence of meningioma.

25. The method of claim 24, wherein a high risk score in the top third tertile compared to a reference scale is indicative of a high risk of local recurrence.

26. The method of claim 24 or 25, wherein the subset comprise at least two genes from each of the following subgroups Group 1-3: Group 1, CDC20, CDK6, CCND2, CKS2, MYBL1, USF1, KIF20A, MDM4, and PIM1; Group 2, CDKN2A, CDKN2C, ARID1B, GAS1, and SPOP; and Group 3, CCN1, COL1A1, FGFR4, IFNGR1, IGF2, KDR, KRT14, MMP9, TAGLN, TMEM30B, and PGK1; and two genes selected from the genes listed in Groups 4-7: Group 4, CHEK1 and MUTYH; Group 5, PGR and ESR; Group 6, LINC02593 and FBLIM1; and Group 7, CCL21 and CD3E.

27. The method of claim 26, wherein the subset comprises at least three genes, or at least four genes, from each subroups Groups 1-3.

28. The method of any one of claims 24 to 27, wherein expression is detected by determining levels of RNA transcripts encoded by the genes.

29. The method of claim 28, determining the level of the RNA transcripts comprises performing an amplification assay, a hybridization assay, a sequencing assay or an array-based hybridization assay.

30. The method of claim 24 or 25, further comprising recommending radiotherapy treatment to the patient when the patient has a high risk score.

31. The method of any one of claims 24 to 27, wherein expression is detected by determining levels of proteins encoded by the genes.

32. The method of claim 31, wherein detecting the level of protein comprises performing an immunoassay.

33. The method of any one of claims 24 to 32, wherein the reference scale is a plurality of risk scores derived from a population of reference patients that have meningioma.

34. The method of any one of claims 24 to 33, wherein the sample from the patient is a tumor tissue sample or a tumor cell sample.

35. A microarray comprising probes for detecting expression of a gene panel for predicting survival, wherein the gene panel consists of the gene ARID1B, CCL21, CCN1, CCND2, CD3E, CDC20, CDK6, CDKN2A, CDKN2C, CHEK1, CKS2, COL1A1, ESR1, EZH2, FBLIM1, FGFR4, GAS1, IFNGR1, IGF2, KDR, KIF20A, KRT14, LINC02593, MDM4, MMP9. MUTYH, MYBL1, PGK1. PGR, PIM1, SPOP. TAGLN, TMEM30B, and USF1, or a subset of at least eight genes of the gene panel; and optionally contains probes for detecting expression of one or more reference genes, wherein the microarray contains probes for detecting no more than 1,000 gene, nor more than 500 genes, nor more than 200 genes, or no more than 100 genes.

36. The microarray of claim 35, wherein the subset comprise at least two genes from each of the following subgroups Group 1-3: Group 1, CDC20, CDK6, CCND2, CKS2, MYBL1, USF1, KIF20A, MDM4, and PIM1; Group 2, CDKN2A, CDKN2C, ARID1B, GAS1, and SPOP; and Group 3, CCN1, COL1A1, FGFR4, IFNGR1, IGF2, KDR, KRT14, MMP9, TAGLN, TMEM30B, and PGK1; and two genes selected from the genes listed in Groups 4-7: Group 4, CHEK1 and MUTYH; Group 5, PGR and ESR; Group 6, LINC02593 and FBLIM1; and Group 7, CCL21 and CD3E.

37. The microarray of claim 36, wherein the subset comprises at least three genes, or at least four genes, from each subroups Groups 1-3.

38. A kit comprising primers and/or probes for detecting expression of a gene panel for predicting survival, wherein the gene panel consists of the gene ARID1B, CCL21, CCN1, CCND2, CD3E, CDC20, CDK6, CDKN2A, CDKN2C, CHEK1, CKS2, COL1A1, ESR1, EZH2, FBLIM1, FGFR4, GAS1, IFNGR1, IGF2, KDR, KIF20A, KRT14, LINC02593, MDM4, MMP9. MUTYH, MYBL1, PGK1. PGR, PIM1, SPOP. TAGLN, TMEM30B, and USF1, or a subset of at least eight genes of the gene panel, and optionally contains primers and/or probes for detecting expression of one or more reference genes.

39. The kit of claim 38, wherein the subset comprise at least two genes from each of the following subgroups Group 1-3: Group 1, CDC20, CDK6, CCND2, CKS2, MYBL1, USF1, KIF20A, MDM4, and PIM1; Group 2, CDKN2A, CDKN2C, ARID1B, GAS1, and SPOP; and Group 3, CCN1, COL1A1, FGFR4, IFNGR1, IGF2, KDR, KRT14, MMP9, TAGLN, TMEM30B, and PGK1; and two genes selected from the genes listed in Groups 4-7: Group 4, CHEK1 and MUTYH; Group 5, PGR and ESR; Group 6, LINC02593 and FBLIM1; and Group 7, CCL21 and CD3E.

40. The kit of claim 39, wherein the subset comprises at least three genes, or at least four genes, from each subroups Groups 1-3.
